(19) United States
(12) Patent Application Publication
(10) Pub. No.: US 2026/0355965 A1
(43) Pub. Date: Nov. 12, 2026

(54) METHODS FOR CONTROLLING AMBIENT-FIELD MOTORS WITHIN AN MRI ENVIRONMENT

(71) Applicant: 4D Surgical, Inc., Salt Lake City, UT (US)

(72) Inventors: Casey James Nordgran, Grantsville, UT (US); Gilbert Ian Montague, Pacifica, CA (US); Blake Edwin Zimmerman, Salt Lake City, UT (US); Lorne Wyatt Hofstetter, Salt Lake City, UT (US)

(21) Appl. No.: 19/546,162

(22) Filed: Feb. 20, 2026

Related U.S. Application Data
(60) Provisional application No. 63/760,909, filed on Feb. 20, 2025.
(60) Provisional application No. 63/894,516, filed on Oct. 6, 2025.

(51) Int. Cl. B25J 9/16; A61B 5/00; A61B 5/055; A61B 34/30; B25J 19/00

(52) U.S. Cl. CPC B25J 9/1679; A61B 5/0036; A61B 5/055; A61B 34/30; B25J 9/163; B25J 19/0075

(57) ABSTRACT

One variation of a method includes: accessing a first mechanical rotor position of a first rotor, in a first ambient-field motor arranged within a first joint a robotic system occupying a bore of an MRI machine, at a first time; accessing a first magnetic field vector of a magnetic field of the MRI machine proximal the first ambient-field motor at the first time; calculating a first mechanical commutation angle for the first ambient-field motor based on the first mechanical rotor position; calculating a first electrical commutation offset for the first ambient-field motor based on the first magnetic field vector; calculating a first composite commutation angle for the first ambient-field motor based on the first mechanical commutation angle and the first electrical commutation offset; and driving the first ambient-field motor according to the first composite commutation angle to traverse the first joint from a first position to a second position.

### CROSS-REFERENCE TO RELATED APPLICATIONS

This Application claims the benefit of U.S. Provisional Application No. 63/894,516, filed on 16 Oct. 2025, and 63/760,909, filed on 20 Feb. 2025, each of which is hereby incorporated in its entirety by this reference.

This Application is related to U.S. patent application Ser. No. 19/390,077, filed on 14 Nov. 2025, which claims priority to U.S. Provisional Application No. 63/840,838, filed on 9 Jul. 2025, and 63/721,025, filed on 15 Nov. 2024, each of which is hereby incorporated in its entirety by this reference.

### TECHNICAL FIELD

This invention relates generally to the field of surgical robotics and, more specifically, to a new and useful methods for controlling ambient-field motors within an MRI environment in the field of surgical robotics.

### BRIEF DESCRIPTION OF THE FIGURES

FIG. 1 is a schematic representation of a robotic system;

FIG. 2 is a schematic representation of one variation of the robotic system;

FIG. 3 is a flowchart representation of a method;

FIG. 4 is a flowchart representation of one variation of the method;

FIG. 5 is a flowchart representation of one variation of the method;

FIG. 6 is a flowchart representation of one variation of the method;

FIG. 7 is a schematic representation of one variation of the robotic system;

FIG. 8 is a schematic representation of one variation of the robotic system;

FIG. 9 is a schematic representation of one variation of the robotic system; and

FIG. 10 is a schematic representation of one variation of the robotic system.

### DESCRIPTION OF THE EMBODIMENTS

The following description of embodiments of the invention is not intended to limit the invention to these embodiments but rather to enable a person skilled in the art to make and use this invention. Variations, configurations, implementations, example implementations, and examples described herein are optional and are not exclusive to the variations, configurations, implementations, example implementations, and examples they describe. The invention described herein can include any and all permutations of these variations, configurations, implementations, example implementations, and examples.

### 1. Method

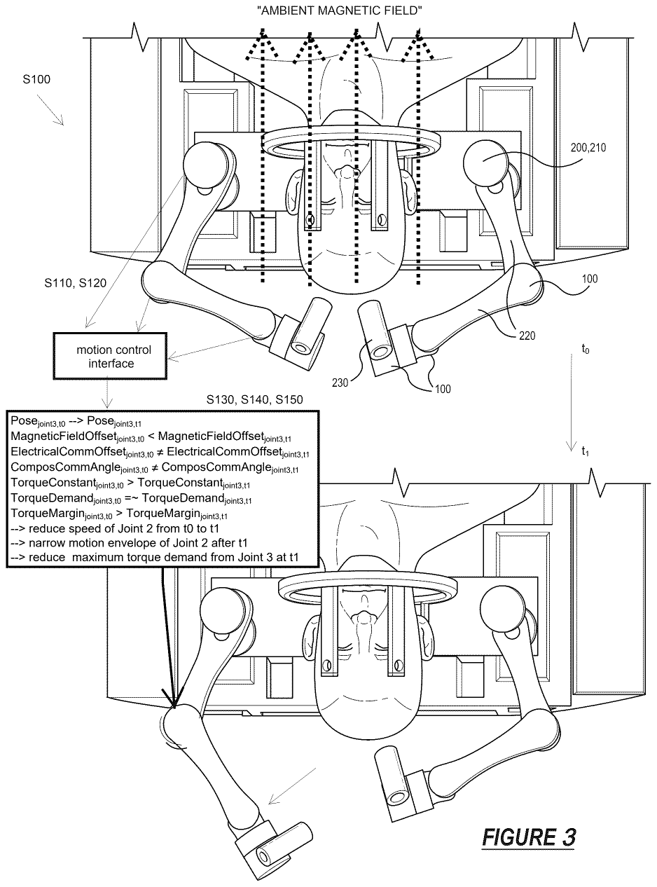

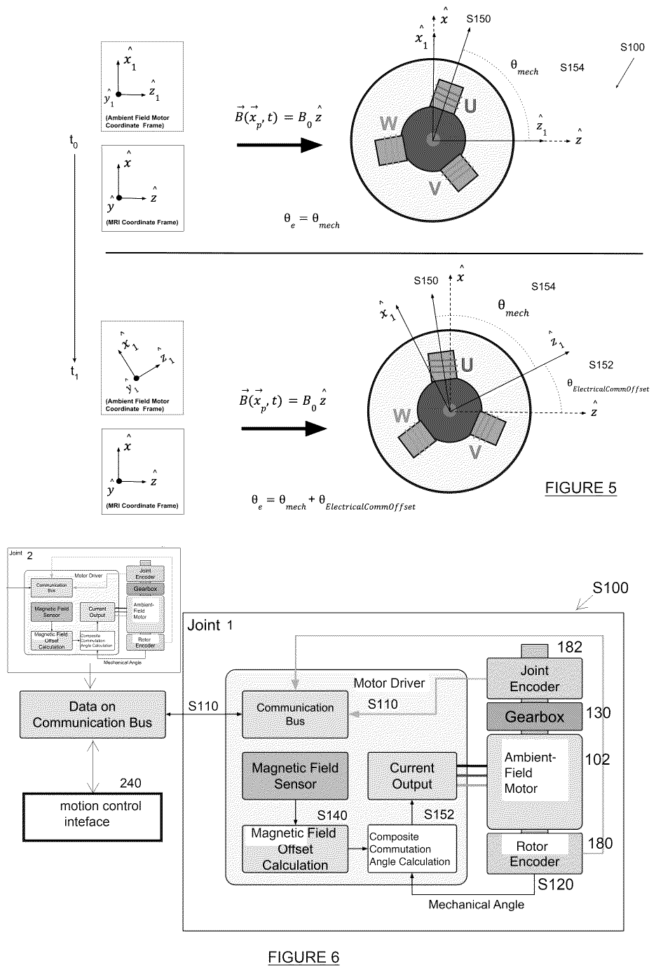

As shown in FIGS. 3-6, a method S100 includes: accessing a first set of joint positions of a set of joints, within a robotic system 200 occupying a bore of an MRI machine (or a magnetic resonance imaging machine, an intraoperative imaging suite), at a first time in Block S110; accessing a first mechanical rotor position of a first rotor, in a first ambient-field motor arranged within a first joint in the set of joints of the robotic system 200, at the first time in Block S120; deriving a first pose of the first ambient-field motor within the MRI machine at the first time based on the first set of joint positions in Block S130; characterizing a first magnetic field offset between the first ambient-field motor and a magnetic field of the MRI machine proximal the first ambient-field motor at the first time based on the first pose of the first ambient-field motor in Block S140; calculating a first composite commutation angle for the first ambient-field motor based on the first mechanical rotor position and the first magnetic field offset in Block S150; and driving the first ambient-field motor according to the first composite commutation angle in Block S160.

### 1.1 Variation: Magnetic Field Vector

One variation of the method S100 includes: accessing a first set of joint positions of a set of joints, within a robotic system 200 occupying a bore of an MRI machine, at a first time in Block S110; accessing a first mechanical rotor position of a first rotor, in a first ambient-field motor arranged within a first joint in the set of joints of the robotic system 200, at the first time in Block S120; deriving a first pose of the first ambient-field motor within the MRI machine at the first time based on the first set of joint positions in Block S130; calculating a first magnetic field vector representing a magnetic field within the bore of the MRI machine proximal the first ambient-field motor at the first time based on the first pose of the first ambient-field motor in Block S140; calculating a first electrical commutation offset for the first ambient-field motor based on the first magnetic field vector in Block S152; calculating a first composite commutation angle for the first ambient-field motor based on the first mechanical rotor position and the first electrical commutation offset in Block S150; and driving the first ambient-field motor according to the first composite commutation angle in Block S160.

### 1.2 Variation: Mechanical Commutation Angle+Electrical Commutation Offset

One variation of the method S100 includes: accessing a first mechanical rotor position of a first rotor, in a first ambient-field motor arranged within a first joint in a set of joints of a robotic system 200 occupying a bore of an MRI machine, at a first time in Block S120; accessing a first magnetic field vector of a magnetic field of the MRI machine proximal the first ambient-field motor at the first time in Block S140; calculating a first mechanical commutation angle for the first ambient-field motor based on the first mechanical rotor position in Block S154; calculating a first electrical commutation offset for the first ambient-field motor based on the first magnetic field vector in Block S152; calculating a first composite commutation angle for the first ambient-field motor based on the first mechanical commutation angle and the first electrical commutation offset in Block S150; and driving the first ambient-field motor according to the first composite commutation angle to traverse the first joint from a first position to a second position in Block S160.

### 2. Applications

The robotic system 200 is configured to perform interventional procedures within a magnetic resonance imaging (MRI) environment. The robotic system 200 includes: a robotic arm 202 arranged and configured to operate within a bore of an MRI machine; and a motion control interface 240—such as arranged outside of the MRI machine—configured to interface the robotic arm with an external controller. The robotic arm: is configured to manipulate an end effector and includes a set of joints, wherein each joint is driven by a corresponding drive unit including an ambient-field motor and a gearbox. The end effector is configured to interact directly with tissue and can include: a needle or syringe for delivery of a therapeutic agent; a drill, reamer, or burr for penetrating hard tissue; a biopsy device for sampling tissue; an ablation device for destroying tissue; or a blade, cutting, or suction tool for tissue resection; etc.

During operation: the MRI machine generates images of a surgical field within its bore; and the robotic arm executes surgical (e.g., neurosurgical) operations inside the bore of the MRI machine according to commanded motions while imaging is ongoing or intermittently active within the bore of the MRI machine.

However, operation within the MRI bore imposes constraints on materials and actuation. For example, the robotic arm can exclude ferromagnetic materials, which may otherwise either: become projectiles within the magnetic field of the MRI machine; and/or distort images generated by the MRI machine. Therefore, the robotic arm can include joints driven by ambient-field motors that exclude internal stator or rotor magnetic elements and rely on the ambient magnetic field generated by the MRI machine for a stator field. Therefore, the MRI machine simultaneously functions as the imaging modality used to guide the surgical procedure and the source of the stator field used by ambient-field motors in the robotic arm to generate torque and actuate the joint in the robotic arm.

While the MRI machine generates a strong and directional magnetic field within its bore, this magnetic field: varies in strength and orientation between the ends and the center of the bore; is fixed relative to the MRI machine but not relative to the robotic arm; and may not be aligned to the rotor axes of ambient-field motors within the robotic arm throughout the entire operating envelope of the robotic arm. In particular, as the robotic arm moves through a series of poses within the bore of the MRI machine, an ambient-field motor may move relative to the magnetic field, thereby changing a magnitude of the magnetic field of the MRI machine that is orthogonal to a rotor rotation axis of the ambient-field motor at the position of the ambient-field motor. The component of the effective stator field thus available to the ambient-field motor to produce useful motor torque may therefore change as a function of the pose of the robotic arm. Therefore, electrical commutation of the ambient-field motor may be affected by its pose (i.e., not only its rotor angular rotation relative to the motor housing), and the torque constant and current-limited torque output capacity of the ambient-field motor may also be affected by its pose.

Therefore, the robotic system 200 can implement Blocks of the method S100 to account for pose-dependent actuation physics of each ambient-field motor in the robotic arm in order to avoid: loss of holding torque at each joint; overshoot or drift from a planned path of the end effector on the robotic arm; oscillation or jitter at a joint, which may be amplified at the end effector; or loss of positional precision of the end effector during image-guided intervention. More specifically, the robotic system 200 can execute Blocks of the method S100 to dynamically calculate electrical commutation parameters for each ambient-field motor, dynamically calculate maximum torques of each ambient-field motor, dynamically constrain motion of each joint in the robotic arm, and/or dynamically update closed-loop control coefficients of each joint during operation of the robotic system 200 (e.g., at a rate of 100 Hz) in order to maintain control authority of the robotic arm.

### 2.1 Commutation

During operation of the robotic system 200 within the bore of the MRI machine, each ambient-field motor magnetically couples to a local magnetic field vector generated by the MRI machine. This coupling between the ambient-field motor and local magnetic field vector depends on: (i) pose of the ambient-field motor within the bore; and (ii) operational state of the MRI machine (e.g., imaging versus non-imaging, gradient configuration). Therefore, an ambient-field motor experiences an effective stator field that is external to the ambient-field motor, fixed relative to the MRI machine rather than the motor housing, and spatially non-uniform across the bore.

Accordingly, electrical commutation of an ambient-field motor depends on two distinct angle components, including: a mechanical commutation angle derived from a mechanical rotor position; and an electrical commutation offset derived from the local magnetic field vector experienced proximal the ambient-field motor and the pose of the ambient-field motor. Therefore, the composite commutation angle for the ambient-field motor can change between time slots even when the rotor does not rotate during these time slots, such as when an upstream joint changes position, when the table shifts, and/or when the operational state of the MRI machine changes.

If the electrical commutation fails to compensate for pose of the ambient-field motor relative to the local magnetic field vector, current supplied to the ambient-field motor is phased away from a torque-producing direction relative to the local magnetic field vector. In this condition: torque output per current input decreases; current demand and heating increase; electromagnetic emissions increase; and joint transient response (e.g., overshoot, damping, settling time) becomes less predictable. These effects can propagate through the robotic arm via corrective activity, resulting in jitter, increased position error, reduced precision at the end effector, unplanned reversal of motor direction, and thus unstable control situations within the robotic arm.

Therefore, the robotic system 200 implements Blocks of the method S100 to derive and apply a pose-and MRI-state-dependent electrical commutation offset for each ambient-field motor in order to derive the composite commutation angle and maintain predictable torque production across poses within the MRI bore. In one implementation, the robotic system 200 derives composite commutation angles based on: rotor encoder positions; joint encoder positions; a pose derived from a kinematic model of the robotic arm; and magnetic field characteristics of the MRI machine represented by a magnetic field model. In this implementation, the robotic system 200 can derive electrical commutation offsets—and thus composite commutation angles—without magnetic field sensors integrated into each joint, thereby reducing mechanical complexity, wiring burden, and electromagnetic noise generated by the robotic arm.

During operation of the robotic system 200 within the bore of the MRI machine, each ambient-field motor: is coupled to the magnetic field generated by the MRI machine; and experiences an effective stator field vector that changes with pose of the ambient-field motor within the MRI bore and an operational state of the MRI machine.

Therefore, the stator field of an ambient-field motor: is external to the ambient-field motor; is essentially spatially fixed relative to the MRI machine rather than to the ambient-field motor; and varies spatially within the MRI bore. As the robotic arm moves within the bore of the MRI machine: the orientation of a rotor axis of the ambient-field motor relative to the stator field may change; and the ambient-field motor may pass between lower-density magnetic fields near the bore openings and higher-density magnetic fields near the center of the bore of the MRI machine. Because the electrical commutation offset of the ambient-field motor is related to the component of the local magnetic field orthogonal to the rotation axis of its rotor, the composite commutation angle of the ambient-field motor may change even if the mechanical angle of the rotor is unchanged. More specifically, the composite (or “total,” “combined”) commutation angle of the ambient-field motor may depend on both rotor mechanical position and a pose-dependent electrical commutation offset derived from the local magnetic field vector experienced by the ambient-field motor.

Generally, if the electrical commutation fails to compensate for pose of the ambient-field motor relative to the local magnetic field vector, the ambient-field motor may apply current out of phase relative to the ambient stator field, which may: increase current demand by the ambient-field motor to achieve a given torque; increase heating within the ambient-field motor; increase electrical and mechanical wear within the ambient-field motor; and reduce the current-limited torque output capacity of the ambient-field motor. Furthermore, incorrect commutation of the ambient-field motor may alter dynamic behavior of the corresponding joint because: effective torque production by the ambient-field motor becomes inconsistent across rotor angle and pose of the ambient-field motor; torque ripple of the ambient-field motor increases; and the joint experiences inconsistent damping and transient response while tracking a command. These effects may further propagate to system-level behavior in the robotic arm because: a first joint producing inconsistent torque output may cause the motion control interface 240 (or other computer system, motor driver, system-level controller, or surgeon interface) to issue larger corrective commands to the corresponding ambient-field motor; such corrective commands increase current draw and heating within the joint; and coupled joints may respond to execution of these larger corrective commands unevenly and thus produce jitter, overshoot, and reduced position precision at the end effector.

Therefore, the robotic system 200 implements Blocks of the method S100 to calculate and apply a pose-dependent electrical commutation offset that: aligns supplied current to the magnetic field vector experienced by an ambient-field motor; and maintains predictable torque production by the ambient-field motor as the ambient-field motor moves through different poses within the MRI bore. In particular, the robotic system 200 can derive an electrical commutation offset for each ambient-field motor in the robotic arm based on: rotor encoder positions; joint encoder positions received from each joint in the robotic arm; a kinematic model of the robotic arm; and known magnetic field characteristics of the MRI machine.

The robotic system 200 can thus derive and implement dynamic electrical commutation offsets for each ambient-field motor without integration of magnetic field sensors within each joint and without directly detecting magnetic field strength and/or direction at each ambient-field motor, thereby reducing mass, mechanical and electrical complexity, and electromagnetic noise generated by the robotic arm.

### 2.2 Torque Constant Variation & Compensation

During operation of the robotic system 200 within the bore of the MRI machine, the torque constant of each ambient-field motor may vary as a function of: pose of the ambient-field motor within the magnetic field of the MRI machine; and operational state of the MRI machine.

Accordingly, a particular current supplied to an ambient-field motor may yield different torque outputs by the ambient-field motor for different poses of the ambient-field motor within the bore of the MRI machine; and a particular torque output demand to the ambient-field motor may require a different current supply to the ambient-field motor for different poses of the ambient-field motor. Therefore, as the robotic arm moves along a path within the bore of the MRI machine, the torque constant of an ambient-field motor may change from the start of the path to the end of the path.

In the robotic arm, the ambient-field motors are mechanically coupled through gearboxes, joints, and arm segments such that motion of one joint generates inertial, gravitational, and constraint-related loads on other joints and ambient-field motors. Therefore, torque demand on a particular ambient-field motor may increase due to motion of upstream or downstream joints in the robotic arm; and the torque output capacity of this ambient-field motor may simultaneously decrease due to pose-dependent variation in its torque constant. If the torque demand of this ambient-field motor approaches or exceeds its torque output capacity, its corresponding joint may lag, slip (or “yield”), or overshoot, and this unintended loss of control authority at the joint may be amplified as lost positional control-within a threshold tolerance-at the end effector as the robotic system 200 attempts to traverse the end effector along a controlled path.

Therefore, rather than actuating a single joint at a time and braking all joints in the robotic arm, the robotic system 200 can execute Blocks of the method S100 to: track pose of each ambient-field motor in the robotic arm; derive a torque constant for each ambient-field motor based on its pose within the bore of the MRI machine and the known magnetic field of the MRI machine; estimate a torque demand for each ambient-field motor based on commanded motion of the robotic arm and system dynamics and kinematics; and calculates a torque margin based on a difference between the torque demand and torque output capacity of each ambient-field motor. Then, if the robotic system 200 detects a torque margin that is approaching or less than a threshold (or safety) margin for a particular ambient-field motor, the robotic system 200 can limit speed, acceleration, jerk, or holding torque of the corresponding joint and/or other joints in the robotic arm in order to increase this torque margin.

The robotic system 200 can thus maintain bounded torque demand relative to available torque output for each ambient-field motor across different poses of the robotic arm within the bore of the MRI machine.

### 2.3 Dynamic Closed-loop Control Coefficients

Because the torque constant of an ambient-field motor defines the relationship between commanded motor current and resulting torque output of the ambient-field motor, changes in torque constant of the ambient-field motor may yield variations in effective plant gain of each ambient-field motor. The robotic system 200 can thus implement Blocks of the method S100 to dynamically update closed-loop control coefficients for the ambient-field motor while its plant gain varies in order to achieve consistent and predictable overshoot, damping characteristics, and settling time in the corresponding joint.

In particular, the robotic system 200 concurrently commands joints in the robotic arm to position the end effector, and ambient-field motors in robotic arm are mechanically coupled through gearboxes, joints, and arm segments. If two joints in the robotic arm exhibit dissimilar transient responses at one time: one joint may rapidly converge on its target position while the second joint slowly converges; corrective actions by the first joint may conflict with convergence of the second joint due to their mechanical coupling; and the end effector may exhibit oscillation, positional error, and increased corrective activity while total current demand, motor temperature, electromagnetic noise, and mechanical wear throughout the robotic arm increases.

Therefore, the robotic system 200 can adjust closed-loop control coefficients (e.g., proportional, integral, and derivative gains) of each joint as a function of: the derived torque constant of the corresponding ambient-field motor (e.g., based on the pose and magnetic field conditions of the ambient-field motor); and the operational state of the MRI machine.

Therefore, by adjusting closed-loop control coefficients of ambient-field motors as a function of their poses within the MRI machine, the robotic system 200 can maintain bounded and predictable overshoot, damping characteristics, and settling time across joints, thereby promoting coordinated convergence of the end effector to its target position or pose without sustained opposing corrective action by the robotic arm.

### 2.4 Motor Architecture+Disclaimer

Generally, the method S100 is described herein as executed in conjunction with three-phase AC synchronous ambient-field gearhead motors that leverage a magnetic field generated by an MRI machine as an external stator field. More specifically, in the ambient-field motor, the effective stator field is supplied externally by the MRI machine such that torque output of the ambient-field motor arises from interaction between rotor-generated magnetic dipoles in the ambient-field motor and the magnetic field generated by the MRI machine.

For example, the ambient-field motor can: leverage a magnetic field already present in the MRI environment; exclude ferromagnetic stator and rotor components, which reduces projectile risk within the MRI machine; and exclude permanent magnets, which reduces projectile risk and MRI image distortion resulting from presence and operation of the robotic system 200 within the magnetic field. The robotic system 200 can drive an ambient-field motor in the robotic arm according to a composite commutation angle by supplying current to a set of coil windings—arranged on the rotor of the ambient-field motor—according to the composite commutation angle in order to generate magnetic dipoles that magnetically couple to the magnetic field of the MRI machine, thereby rotating the rotor, driving a gearbox within the corresponding joint, and articulating the corresponding joint. More specifically, during operation, the robotic system 200 supplies current to the set of coil windings within the ambient-field motor according to the composite commutation angle derived for the ambient-field motor (e.g., during a current or preceding time slot); the energized coil windings generate magnetic dipoles; and the magnetic dipoles magnetically couple to the magnetic field of the MRI machine. Lorentz forces are generated on the coil windings via this coupling such that the rotor experiences torque about its rotational axis, which drives the gearbox within the joint and articulates the joint.

Thus, the robotic system 200 is described herein as executing Blocks of the method S100 to control three-phase AC synchronous ambient-field gearhead motors in which three alternating currents are supplied to three radially offset coil windings to achieve accurate electrical commutation of the ambient-field motor based on a composite commutation angle. The robotic system 200 can thus: achieve smooth torque production by the ambient-field motor; limit torque ripple in the ambient-field motor; and implement commutation behavior compatible with established motor-drive architectures while accounting for pose-dependent electrical commutation offsets at each ambient-field motor.

Additionally or alternatively, the robotic system 200 can operate ambient-field motors characterized by other winding topologies or drive schemes, such as single-phase ambient-field motors; or ambient-field motors including more or fewer than three phases. For example, the robotic system 200 can implement current-mode control, voltage-mode control, or hybrid control scheme techniques. Similarly, the robotic system 200 can implement methods and techniques described herein with different commutation strategies, such as field-oriented control (FOC); trapezoidal commutation; or other schemes that apply phase-dependent excitation to motor windings.

Additionally or alternatively, the robotic system 200 can implement similar methods and techniques to control direct-drive (i.e., non-geared) ambient-field motors.

Furthermore, Blocks of the method S100 is described herein as executed by the motion control interface 240. However, Blocks of the method S100 can be additionally or alternatively executed locally by an ambient-field motor itself, a system-level controller, and/or a surgeon interface.

### 2.5 Terms

Magnetic field offset herein refers to an angular relationship between a rotor axis of a rotor of an ambient-field motor and the direction a local magnetic field vector—generated by the MRI machine—proximal (e.g., at a center of the rotor of) the ambient-field motor at a given time. The magnetic field offset varies as a function of: pose of the ambient-field motor within the bore of the MRI machine; and/or an operational state of the MRI machine.

Mechanical commutation angle herein refers to an angular position of a rotor of an ambient-field motor about its rotational axis, such as derived from an output of a rotor encoder coupled to the rotor. The mechanical commutation angle represents the instantaneous mechanical orientation of the rotor relative to a reference frame fixed to a housing of the ambient-field motor and is independent of the magnetic field of the MRI machine. The mechanical commutation angle may be expressed as an absolute angular position or as a phase angle scaled according to a quantity of electrical pole pairs of the ambient-field motor.

Electrical commutation offset herein refers to an angular offset between: a reference orientation associated with the rotor of an ambient-field motor; and a magnetic field vector of the MRI machine proximal the ambient-field motor. The electrical commutation offset represents a pose-dependent phase adjustment that aligns commanded phase currents within the coil windings of the ambient-field motor to a torque-producing direction relative to the local magnetic field vector. The electrical commutation offset may vary as a function of: pose of the ambient-field motor within the bore of the MRI machine; table position; and/or operational state of the MRI machine; etc.

Composite commutation angle herein refers to a combination of the mechanical commutation angle and the concurrent electrical commutation offset. The composite commutation angle is implemented by the motor control interface (or other motor driver) to electronically switch current between the motor windings within an ambient-field motor to create a magnetic field within the ambient-field motor that produces continuous rotation and/or precise torque control of the ambient-field motor.

Torque constant herein refers to a relationship between current supplied to an ambient-field motor and torque generated by the ambient-field motor under a local magnetic field condition. The torque constant may vary as a function of pose of the ambient-field motor within the MRI machine and operational state of the MRI machine.

Torque output limit herein refers to a maximum torque that an ambient-field motor can generate at a given time based on its torque constant and a current limit of the ambient-field motor.

Torque margin herein refers to a difference between an actual or estimated torque demand of an ambient-field motor and its concurrent torque output limit.

Magnetic field model herein refers to a representation of magnetic field magnitude and orientation within the bore of an MRI machine, such as including spatial non-uniformities and state-dependent magnetic field variations.

Pose herein refers to a position and orientation of an ambient-field motor or joint within a coordinate frame associated with the robotic system, the MRI table, or the MRI machine, etc.

Motor driver herein refers to a printed circuit assembly with electronic components capable of providing power to the motor phases.

### 3. Drive Unit+Ambient-Field Motor

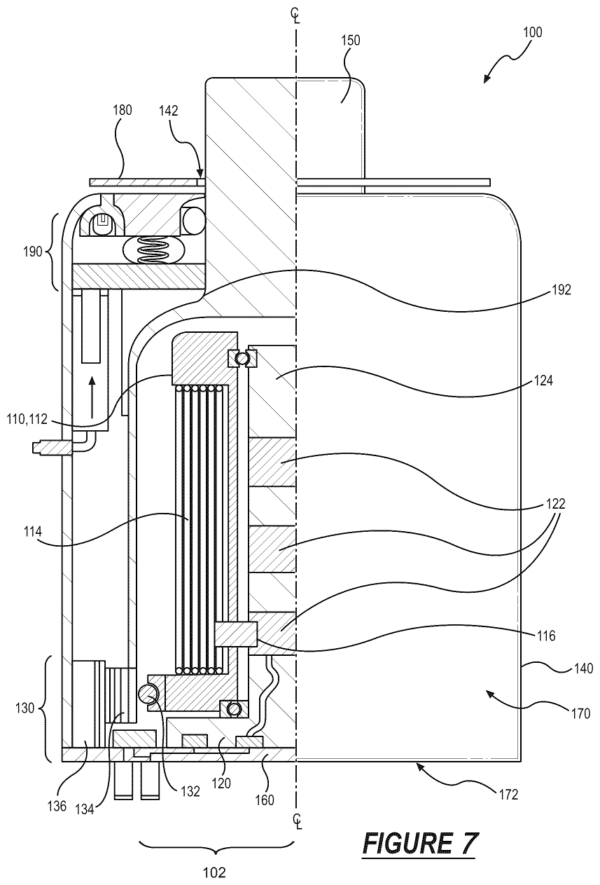

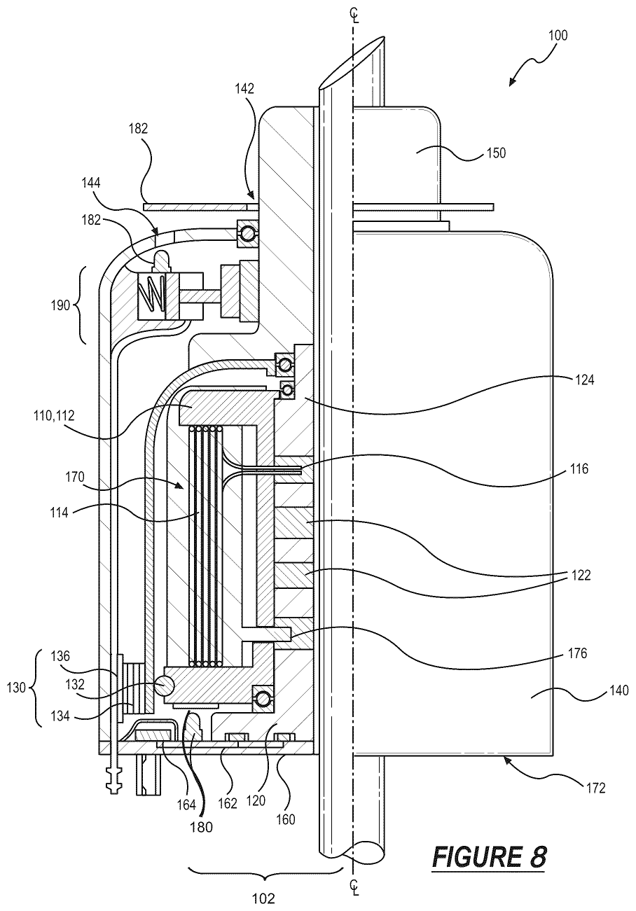

In one variation shown in FIGS. 7-10, a drive unit 100 defines a statorless gearhead ambient-field motor including both an ambient-field motor and a gearbox. In particular, the drive unit 100 can include: a rotor 110; a slip-ring assembly 120; a power transmission 130 coupled to the rotor 110; a non-conductive driveshaft 150; and a non-ferrous shield.

The rotor 110: is configured to rotate about a rotor axis; and includes a set of coil windings 114 configured to generate magnetic dipoles that interact with a magnetic field generated by a magnetic resonance imaging machine to rotate the rotor 110 about the rotor axis.

The slip-ring assembly 120: is coaxial with the rotor axis; and is configured to electrically couple to the set of coil windings 114.

The power transmission 130 is coupled to the rotor 110.

The non-conductive driveshaft 150: is coupled to the power transmission 130; and is configured to output torque generated by the set of coil windings 114 interacting with the magnetic field generated by the magnetic resonance imaging machine and transmitted from the rotor 110 to the non-conductive driveshaft 150 by the power transmission 130.

The non-ferrous shield: is arranged about the set of hollow-core coil windings 114; and is configured to attenuate propagation of electromagnetic fields, generated by the set of hollow-core coil windings 114, toward the magnetic resonance imaging machine.

### 3.5 Variation: Driveshaftless Statorless Gearhead Ambient-field Motor

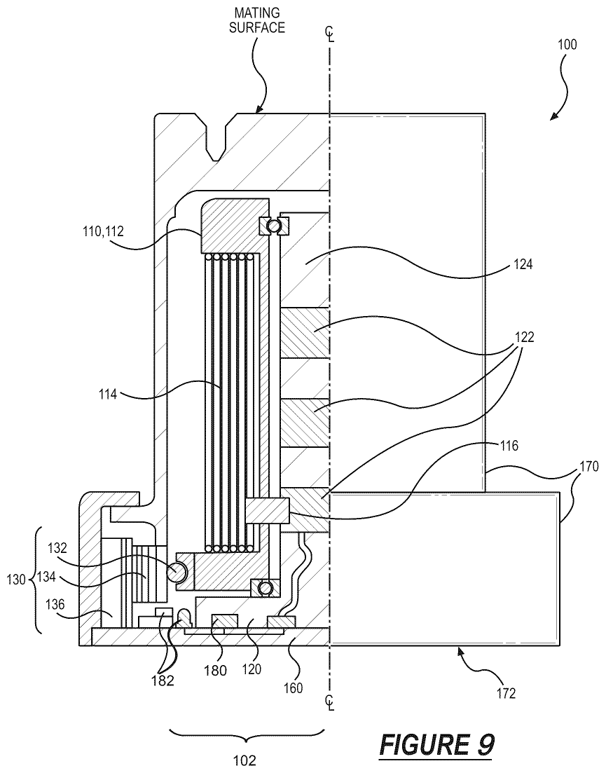

In one variation shown in FIG. 9, the power transmission 130: is coupled to the rotor 110; and is configured to output torque generated by the set of coil windings 114 interacting with the magnetic field generated by the magnetic resonance imaging machine and transmitted from the rotor 110 into the power transmission 130.

### 3.6 Variation: Internal Slip-ring Assembly

In one variation shown in FIGS. 7, 8, and 9, the rotor 110: is configured to rotate about a rotor axis; and includes a set of coil windings 114 configured to generate magnetic dipoles that interact with a magnetic field generated by a magnetic resonance imaging machine to rotate the rotor 110 about the rotor axis.

The slip-ring assembly 120: is coaxial with the rotor axis; extends into a center of the rotor 110; and includes a set of slip-rings 122 configured to electrically couple to the set of coil windings 114.

The power transmission 130 is coupled to the rotor 110.

The housing 140: contains the rotor 110, the slip-ring assembly 120, and the power transmission 130; and defines a driveshaft aperture 142.

The non-conductive driveshaft 150: is coupled to the power transmission 130; extends through the driveshaft aperture 142 of the housing 140; and is configured to output torque generated by the set of coil windings 114 interacting with the magnetic field generated by the magnetic resonance imaging machine and transmitted from the rotor 110 to the non-conductive driveshaft 150 by the power transmission 130.

### 3.7 Variation: Strain-Wave Power Transmission

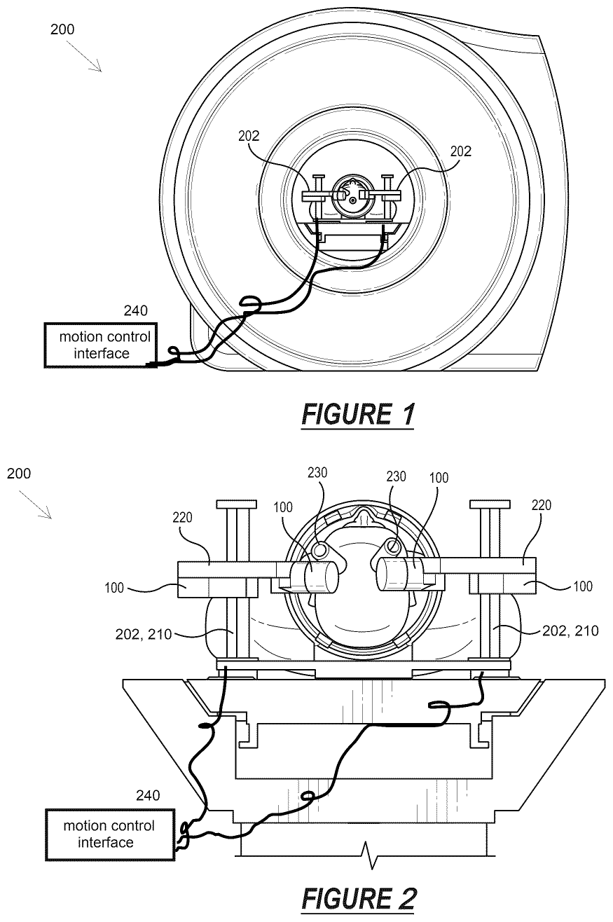

In another variation shown in FIGS. 1, 2, and 10, the rotor 110: is configured to rotate about a rotor axis; includes a set of hollow-core coil windings 114 configured to generate magnetic dipoles that interact with a magnetic field generated by a magnetic resonance imaging machine to rotate the rotor 110 about the rotor axis; and includes a set of rotor contacts 116 electrically coupled to the set of hollow-core coil windings 114 and extending toward the rotor axis.

The slip-ring assembly 120: is coaxial with the rotor axis; is arranged within the rotor 110; and includes a set of slip-rings 122 configured to electrically couple to the set of rotor contacts 116.

The strain-wave transmission 130 includes: a wave-generator bearing 132 coupled to the rotor 110 and laterally adjacent the set of hollow-core coil windings 114; a flexspline cup 134 arranged about the wave-generator bearing 132 and configured to rotate about the rotor axis; and a circular-spline ring 136 arranged about the flexspline cup 134 and coaxial with the rotor 110.

The housing 140: contains the rotor 110, the slip-ring assembly 120, the wave-generator bearing 132, and the flexspline cup 134; and defines a driveshaft aperture 142.

The non-conductive driveshaft 150: is coupled to the strain-wave transmission 130; extends through the driveshaft aperture 142 of the housing 140; and is configured to output torque a) generated by the set of hollow-core coil windings 114 interacting with the magnetic field generated by the magnetic resonance imaging machine and b) transmitted from the rotor 110 to the non-conductive driveshaft 150 by the strain-wave transmission 130.

### 3.8 Drive Unit Operation

Generally, a drive unit 100: defines a statorless gearhead ambient-field motor configured to actuate joints or other elements within a surgical robotic system 200 (e.g., a robotic arm 202) operating within an MRI machine; and includes specific classes of non-ferrous, non-conductive, and non-magnetic components in a particular arrangement to limit degradation of imaging quality of the MRI machine in order to enable concurrent surgical operations and imaging within the MRI machine, such as imaging-assisted neurosurgery.

In particular, in order to limit imaging quality degradation of the MRI machine resulting from presence and/or operation of the drive unit 100 within the MRI machine (given the output torque rating of the drive unit 100), the drive unit 100 can include: a first electromagnetic shield 170 that attenuates electromagnetic radiation generated by coil windings 114, a rotor encoder 180, a joint encoder 182, a motor driver 164, wiring, and other electrical components within the drive unit 100; perforations in the first electromagnetic shield 170 that interrupt Eddy-current loops that may otherwise distort the magnetic field generated by the MRI machine and/or induce emission of secondary electromagnetic radiation; and a non-conductive (e.g., polymer) output shaft that-while approximating the geometry of an antenna-reradiates relatively little or no radio-frequency noise generated within the housing 140 due to operation of the statorless gearhead ambient-field motor and/or due to the magnetic field generated by the MRI machine.

Furthermore, the drive unit 100 includes limited volumes of ferrous, conductive, and magnetic materials (given the output torque rating of the drive unit 100) in order to limit distortion of images generated by the MRI machine. In particular, the drive unit 100 can exclude a stator (e.g., permanent magnets) and can instead leverage the magnetic field generated by the MRI machine for the stator field with which coil windings 114 in the drive unit 100 interact in order to rotate the rotor 110, the power transmission 130, and the non-conductive driveshaft 150. The drive unit 100 can also include: hollow-core coil windings 114 arranged on the rotor 110; and slip-rings 122 with non-magnetic, smooth contacts that communicate current to these hollow-core coil windings 114, thereby eliminating a traditional commutator and brush assembly that may arc, exhibit inductive heating, and emit radio-frequency noise in the presence of the magnetic field of the MRI machine.

Iron or conductive coil winding cores may both: generate Eddy currents that oppose and distort intended magnetic fields of the MRI machine; exhibit inductive heating in the presence of the magnetic field generated by the MRI machine. Therefore, exclusion of such cores may reduce distortion of images generated by the MRI machine and eliminate a source of heat within the drive unit 100 during operation, thereby: reducing cogging and smoothing a torque output profile of the statorless gearhead ambient-field motor; and enabling incorporation of less thermally-resilient or stable materials-that also produce less distortion of and interaction with the magnetic field generated by the MRI machine-within the drive unit 100, such as one or more polymers that form the non-conductive driveshaft 150, the housing 140, a rotor hub 112, a column 124 of the slip-ring assembly 120, and/or elements of the power transmission 130.

Furthermore, the drive unit 100 can eliminate elements (e.g., a traditional rotor shaft), nest elements (e.g., a slip-ring assembly 120, rotor 110, and gearbox), and/or consolidate traditionally distinct elements (e.g., a housing 140 and a circular-spline ring 136 of a strain-wave gearbox; a drive unit 100 base 160 and a motor driver 164) in order to: reduce total mass and volume of the drive unit 100 (for the output torque rating of the drive unit 100), thereby reducing torque rating and braking requirements of other drive units 100 within the robotic arm 202; and reduce the volume of the drive unit 100, thereby reducing the surface area of the housing 140, reducing the surface area and total mass of the first electromagnetic shield 170, and thus reducing a vector for Eddy-current generation and electromagnetic noise transmission within the drive unit 100.

### 3.9 Example: Strain-Wave Transmission With Integral Housing and Shield

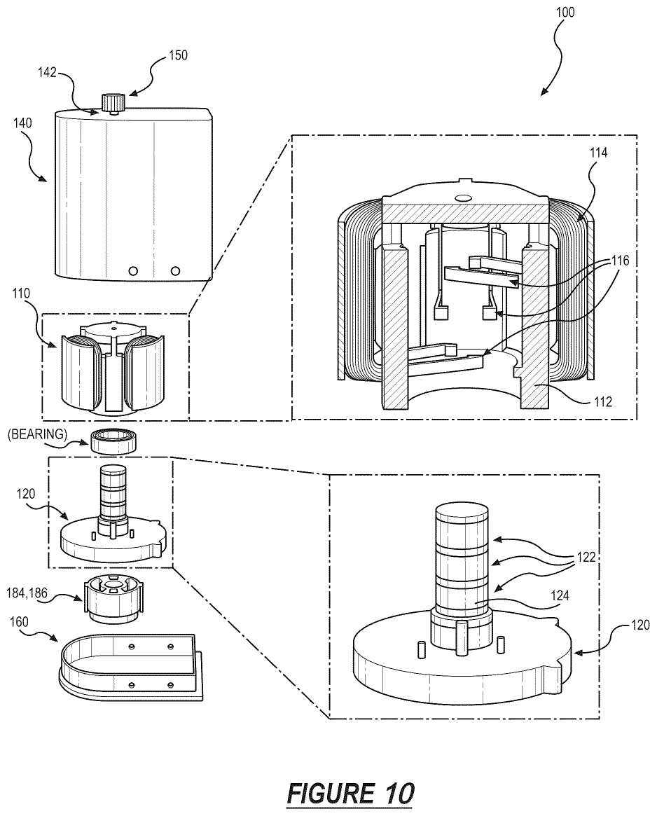

In one example shown in FIG. 10, the slip-ring assembly 120 includes a column 124 (e.g., a polymer “post”) supporting a set of slip-rings 122. The rotor 110 includes: a polymer rotor hub 112 supported on the column 124 of the slip-ring assembly 120, such as by a pair of ceramic ball bearings or bushings; and a set of (e.g., three, six) non-ferrous (e.g., copper) coil windings 114 radially arranged about and mounted to the rotor hub 112.

In this example, the power transmission 130 defines a strain-wave transmission 130 including: a wave-generator bearing 132; a flexspline cup 134; and a circular-spline ring 136. The wave-generator includes an elliptical plug arranged on, integrated into, or physically coextensive with (i.e., of the same, continuous material) the rotor hub 112 of the rotor 110 and adjacent the set of coil windings 114. For example, the elliptical plug can include a set of bearings or a bearing surface encircling or extending outwardly between the set of coil windings 114.

In this example, the flexspline cup 134: defines a bellform (or “bell-shaped”) geometry; is arranged about the wave-generator bearing 132; is configured to rotate about the rotor axis; includes a set of external gear teeth; is configured to elastically deform into a rotating elliptical cross-section that follows the wave-generator bearing 132; encapsulates the sides and tops of the wave-generator bearing 132, the rotor 110, and the slip-ring assembly 120; and is directly coupled (e.g., fastened, bonded) to the non-conductive driveshaft 150, which passes through and is supported by a bearing or journal in the driveshaft aperture 142 in the housing 140.

The circular-spline ring 136: defines a bellform geometry nested over the flexspline cup 134; is arranged about the flexspline cup 134 and coaxial with the rotor 110; defines a set of internal gear teeth configured to mesh with the external gear teeth of the flexspline cup 134; and forms the housing 140 that defines the driveshaft aperture 142 and encapsulates the sides and tops of the flexspline cup 134, the wave-generator bearing 132, the rotor 110, and the slip-ring assembly 120.

In this example, the first electromagnetic shield 170 (e.g., a copper-alloy mesh) is applied (e.g., bonded, plated, sputtered, and/or etched) directly to the outer surface of the circular-spline ring 136.

In this example, the drive unit 100 further includes a base 160 formed by a printed circuit board (or “PCB”): that rigidly couples the slip-ring assembly 120 to the circular-spline ring 136, such as via a solder joint and threaded fasteners, respectively; supports a motor driver 164; includes a set of traces 162 that electrically couple the motor driver 164 to the slip-ring assembly 120; and includes a conductive trace layer extending across the (outer) surface of base 160 to form a second electromagnetic shield 172 electrically coupled to the first electromagnetic shield 170 arranged on the circular-spline ring 136. These first electromagnetic shields 170 on the circular-spline ring 136 and the base 160 can thus form a continuous return path and/or a common reference potential that suppression transmission of electromagnetic noise from the drive unit 100.

Thus, in this implementation, the slip-ring assembly 120, the rotor 110 and wave-generator bearing 132 assembly, and the flexspline cup 134 are coaxial and nested within the circular-spline ring 136, which also functions as the housing 140. The base 160 encloses the slip-ring assembly 120, the rotor 110 and wave-generator bearing 132 assembly, and the flexspline cup 134 within the circular-spline ring 136 and functions to locate the slip-ring assembly 120 relative to the circular-spline ring 136, to communicate electrical signals from the motor driver 164 to the slip-ring assembly 120, and to complete the first electromagnetic shield 170 around the drive unit 100.

Thus, in this example, the drive unit 100 can exclude distinct housing 140 and rotor shaft components, and other components can perform multiple functions traditionally allocated to discrete components, thereby reducing electromagnetic footprint, mass, and volume of the drive unit 100 for a given torque rating of the drive unit 100.

### 4. Multi-Joint Intra-MRI Robotic Arm

As shown in FIGS. 1-3, multiple similar or identical drive units 100 can be combined with a robotic arm base 210, a set of robotic arm segments 220, and an end effector 230 to form a multi-joint surgical robotic arm 202. In particular, the robotic arm 202 can include one instance of the drive unit 100: in the robotic arm base 210 of the multi-joint surgical robotic arm 202; at each arm joint of the multi-joint surgical robotic arm 202; and in the end effector 230.

Because the drive unit 100 is configured to yield limited electromagnetic noise and includes minimal conductive, metallic, and/or ferrous material, the multi-joint surgical robotic arm 202 may be operated within a primary (e.g., Bo) magnetic field generated by the MRI machine-such as to perform live, imaging-assisted neurosurgery actions on a patient within the MRI machine-while the MRI machine captures live (brain) scans of the patient, which may be served to a surgeon remotely-controlling the multi-joint surgical robotic arm 202, such as within or outside of the MRI machine.

Furthermore, multiple similar or identical surgical robotic arms 202 can be arranged within the MRI machine, such as on each side of a sagittal plane of the table in the MRI machine and near a head of the table.

### 4.1 Robotic Arm Configuration

Generally, multiple similar or identical instances of the drive unit 100 can be integrated into joints of a robotic arm 202-including a robotic arm base 210, a robotic arm segment 220, and an end effector 230-to articulate elements of the robotic arm 202, as shown in FIGS. 1-3.

In one implementation, the rotor 110, the slip-ring assembly 120, the strain-wave transmission 130, the housing 140, and the non-conductive driveshaft 150 collectively define a first drive unit 100 (or “statorless gearhead ambient-field motor”) interposed between the robotic arm base 210 and the robotic arm segment 220 and configured to drive the robotic arm segment 220 over a first range of positions on the robotic arm base 210.

In this implementation, the robotic arm 202 can further include a second drive unit 100 interposed between the robotic arm segment 220 and the end effector 230 and configured to drive the end effector 230 over a second range of positions on the robotic arm segment 220. The second drive unit 100 can include a second rotor: configured to rotate about a second rotor axis; including a second set of hollow-core coil windings 114 configured to generate magnetic dipoles that interact with the magnetic field generated by the magnetic resonance imaging machine to rotate the second rotor about the second rotor axis; and including a second set of rotor contacts 116 electrically coupled to the second set of hollow-core coil windings 114 and extending toward the second rotor axis. The second drive unit 100 can further include a second slip-ring assembly 120: coaxial with the second rotor axis; arranged within the second rotor; and including a second set of slip-rings 122 configured to electrically couple to the second set of rotor contacts 116. The second drive unit 100 can also include a second strain-wave transmission 130: including a second wave-generator bearing 132 coupled to the second rotor and laterally adjacent the second set of hollow-core coil windings 114; a second flexspline cup 134 arranged about the second wave-generator bearing 132 and configured to rotate about the second rotor axis; and a second circular-spline ring 136 arranged about the second flexspline cup 134 and coaxial with the second rotor.

The second drive unit 100 can further include a second housing 140: containing the second rotor, the second slip-ring assembly 120, the second wave-generator bearing 132, and the second flexspline cup 134; and defining a second driveshaft aperture 142. A second non-conductive driveshaft 150: is coupled to the second strain-wave transmission 130; extends through the second driveshaft aperture 142 of the second housing 140; and is configured to output torque a) generated by the second set of hollow-core coil windings 114 interacting with the magnetic field generated by the magnetic resonance imaging machine and b) transmitted from the second rotor to the second non-conductive driveshaft 150 by the second strain-wave transmission 130.

Thus, in this implementation, each instance of the drive unit 100 (i.e., each drive unit 100) can operate independently or in coordination to position the end effector 230 within the MRI machine while limiting emission of magnetic fields and radio-frequency noise that may degrade imaging by the MRI machine. Because each drive unit 100 includes a high-gear-reduction power transmission 130, including a brake 190 acting directly on its non-conductive driveshaft 150, and leverages the magnetic field generated by the magnetic resonance imaging machine for its stator field, the robotic arm 202 can execute precise, low-noise motions within the imaging bore of the MRI machine without introducing (substantive masses of) ferrous or magnetic materials or radio-frequency interference within the magnetic field of the MRI machine. The non-conductive driveshafts 150, electromagnetic shielding, and non-ferrous structural materials of each drive unit 100 can also limit generation of Eddy currents within the drive units 100 and thus limit distortion of the magnetic field of the MRI machine during imaging sequences.

### 5. Motion Control Interface+Inter-Joint Communications

As shown in FIGS. 1-3, the robotic system 200 includes a motion control interface 240 arranged outside of the bore of the MRI machine and interposed between the robotic arm and a system-level controller and surgeon interface. In particular, the motion control interface 240 operates outside of the high-field MRI environment and maintains electrical and data communication with the motor driver of each ambient-field motor arranged within the joints of the robotic arm. In one example, the motor driver within each ambient-field motor communicates with the motion control interface 240 over a wired electrical or optical communication link, such as a fieldbus, that conveys motor state information and control parameters between the robotic arm and the motion control interface 240. For example, the motion control interface 240 and the ambient-field motors can communicate over: a daisy-chain differential pair, shielded twisted pair, fiber, isolated serial connection; EtherCAT or Ethernet; or a galvanic isolation boundary; etc.

The motion control interface 240 can thus: receive data from each ambient-field motor in the robotic system 200, including mechanical rotor positions of the ambient-field motors and joint positions of corresponding joints of the robotic arm; and derive a current pose of each ambient-field motor within the MRI machine based on these data and a stored kinematic model of the robotic arm. The motion control interface 240 can then: access a magnetic field model that represents the magnetic field strengths and directions within the MRI machine; transform a pose of each ambient-field motor into a corresponding magnetic field vector proximal the ambient-field motor based on this magnetic field model. The motion control interface 240 can then calculate a composite commutation angle for each ambient-field motor in the robotic arm based on: the mechanical rotor position of the ambient-field motor; and this corresponding magnetic field vector proximal the ambient-field motor.

The motion control interface 240 can then pass these composite commutation angles to corresponding motor drivers within each joint or external to the robotic arm, which then drive the ambient-field motors according to these pose-dependent composite commutation angles.

The motion control interface 240 further interprets torque demand and torque margin of each ambient-field motor (or joint more generally) in the robotic arm based on the kinematic model of the robotic arm and real-time motion of the robotic arm; and then adjust speed, velocity, acceleration, jerk, and/or envelope limits and set brake status for each ambient-field motor and joint in order to maintain predictable motion and avoid loss of control authority at any joint in the robotic arm during operation. The motion control interface 240 can communicate these motion limits, envelope limits, and brake statuses, etc. to the system-level controller and/or surgeon interface.

Therefore, the motion control interface 240 can coordinate motion of multiple joints of the robotic arm simultaneously while accounting for pose-dependent actuation physics imposed by operation of the robotic arm within the magnetic field generated by the MRI machine.

### 6. Magnetic Field Model

During operation of the robotic system 200 within the bore of the MRI machine: ambient-field motors within the robotic arm rely on the MRI magnetic field as an external stator field; and electrical commutation offsets, torque constants, motion envelopes, and closed-loop control behavior vary with the local magnetic field vector experienced by each ambient-field motor.

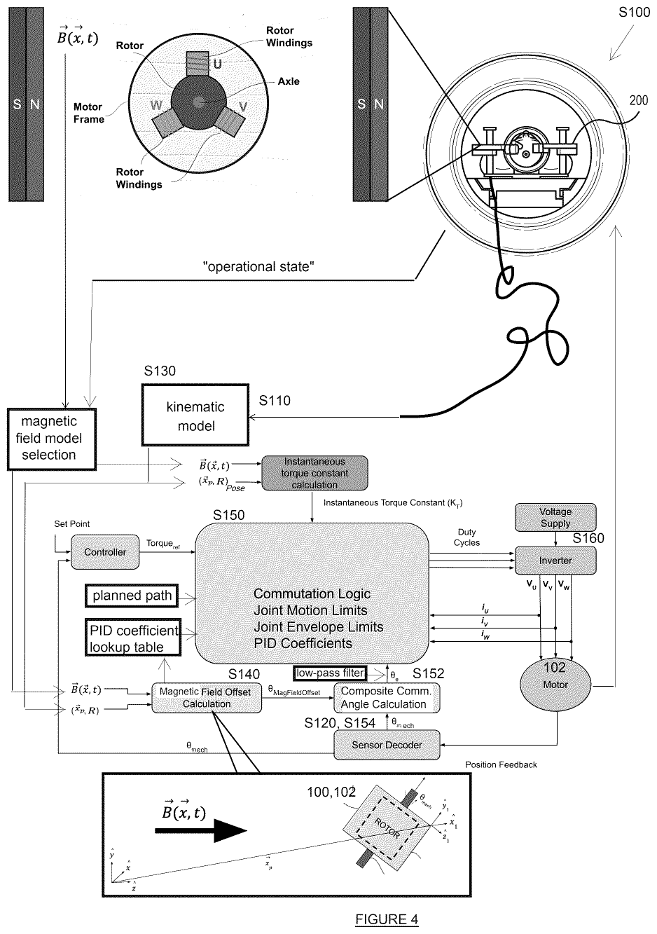

Therefore, the robotic system 200 references a representation of the magnetic field within the MRI bore as shown in FIG. 4 to: derive pose-dependent electrical commutation offsets for ambient-field motors within the robotic arm; derive pose-and MRI-state-dependent torque constants; constrain speed, acceleration, and position envelopes across joints; and adjust closed-loop control coefficients to maintain predictable joint response during actuation. For example, the motion control interface 240 can reference the magnetic field representation in the form of: a magnetic field map including spatially distributed magnetic field vectors; a parameterized magnetic field model; or a combination of measured field data and inferred field behavior.

Thus, the robotic system 200 can reference the magnetic field model that represents: magnetic field magnitudes, directions, non-uniformities within and near the ends of the bore of the MRI machine; and the effective magnetic field present around the rotor of an ambient-field motor over a range of poses of the robotic arm when occupying the bore of the MRI machine.

### 6.1 Preloaded Magnetic Field Representation

In one implementation: the motion control interface 240 accesses a preloaded magnetic field representation associated with the MRI machine, such as supplied by an MRI supplier; generated during installation or commissioning of the MRI machine; and/or derived from prior characterization of the MRI machine by the robotic system 200, as described below.

### 6.2 In-Situ Magnetic Field Mapping and Calibration

In one variation, during a setup period prior to a surgical procedure, the robotic system 200 generates a magnetic field model of the MRI machine based on in situ measurements collected by the robotic arm.

In particular, during a setup period prior to the surgery, the robotic system 200 can: navigate the robotic arm along a calibration path within the bore of the MRI machine based on a baseline magnetic field model of the MRI machine; record a series of magnetic field vectors based on outputs of a magnetic field sensor, arranged on the robotic system 200, during navigation of the robotic arm along the calibration path; and construct a three-dimensional magnetic field model of the MRI machine based on the series of magnetic field vectors.

### 6.2.1 Example: Scalar Magnetic Field Strength Assumption

In one implementation, during the setup period, the robotic system 200 can: set narrow speed, acceleration, and jerk limits of each ambient-field motor within the robotic arm; load a scalar magnetic field magnitude estimate for the magnetic field of the MRI machine; traverse the robotic arm through a sequence of poses within the bore of the MRI machine according to these motion limits and based on an assumption of the scalar magnetic field magnitude estimate parallel to the axis of the bore of the MRI machine; and collect magnetic field magnitude and orientation data at each pose via a magnetic field sensor arranged on the robotic arm, such as within a connected end effector or arranged in a distal joint of the robotic arm. The robotic system 200 then: assembles these magnetic field magnitude and orientation data into a sparse magnetic field map defined in a coordinate frame of the robotic arm; and interpolates between these magnetic field strengths to derive a three-dimensional magnetic field map representing magnetic field gradients within the bore of the MRI machine.

The robotic system 200 can thus generate a magnetic field map that: uniquely represents the MRI machine and the state of the magnetic field of the MRI machine immediately before a surgery; and/or represents effects that a patient and/or the robotic system 200 within the bore of the MRI machine may impart on this magnetic field. Therefore, by generating (or calibrating) the magnetic field model prior to a surgical procedure, the robotic system 200 can: reduce commutation offset derivation errors during the surgery; reduce torque constant derivation errors; define more accurate motion limits; preserve and expand control authority of the robotic arm; and increase positional accuracy, holding, and speed of the robotic arm during operation.

### 6.2.2 Example: Line Mapping

In another implementation, the robotic arm includes a three-axis magnetic field sensor, such as arranged on a distal end of the robotic arm or within an end effector coupled to the distal end of the robotic arm for the setup period.

The robotic system 200 then: drives each ambient-field motor in the robotic arm to locate the magnetic field sensor at a first lateral and vertical position over the bed of the MRI machine; triggers the bed to traverse a longitudinal distance within the MRI machine bore; implements line-scanning techniques to record a series of magnetic field magnitudes and directions (i.e., magnetic field vectors) as the bed traverses this longitudinal distance; and repeats this process for a set of unique lateral and vertical positions of the magnetic field sensor.

The robotic system 200 then: assembles these magnetic field line scans into a sparse magnetic field map; and interpolates between these magnetic field scan lines to generate the three-dimensional magnetic field model.

### 6.2.3 MRI-State-Specific Magnetic Field Models

In this variation, the robotic system 200 can implement similar methods and techniques to generate multiple three-dimensional magnetic field models, including one magnetic field model per imaging state of the MRI machine. More specifically, the total magnetic field generated by the MRI machine—and thus exposed to an ambient-field motor in the robotic arm—may differ between imaging and non-imaging states (or gradient configurations) of the MRI machine.

Therefore, the robotic system 200 can implement the foregoing methods and techniques to: generate a first three-dimensional magnetic field model when the MRI machine occupies a non-imaging state; and generate a second, different three-dimensional magnetic field model when the MRI machine occupies an imaging state.

Later, the motion control interface 240 can: receive (e.g., pull) a current operational state from the MRI machine; select a magnetic field model—from memory—associated with the current operational state from the MRI machine; and implement this particular magnetic field model to derive commutation offsets, torque constants, and/or closed-loop control coefficients for ambient-field motors in the arm, such as until the state of the MRI machine changes.

### 6.2.4 Localized Magnetic Field Mapping

Additionally or alternatively, the robotic system 200 can selectively map the magnetic field of a particular region of the bore containing the planned envelope of the robotic arm during the upcoming surgery, such as rather than map the entire bore of the MRI machine, in order to reduce the duration of the setup period.

### 7. Commutation

As shown in FIGS. 3-5, the motion control interface 240 can: access a first set of joint positions of a set of joints, within a robotic system 200 occupying a bore of an MRI machine, at a first time in Block S110; access a first mechanical rotor position of a first rotor, in a first ambient-field motor arranged within a first joint in the set of joints of the robotic system 200, at the first time in Block S120; derive a first pose of the first ambient-field motor within the MRI machine at the first time based on the first set of joint positions in Block S130; characterize a first magnetic field offset between the first ambient-field motor and a magnetic field of the MRI machine proximal the first ambient-field motor at the first time based on the first pose of the first ambient-field motor in Block S140; and calculate a first composite commutation angle for the first ambient-field motor based on the first mechanical rotor position and the first magnetic field offset in Block S152. The motion control interface 240 (or other computer system, motor driver, system-level controller, or surgeon interface) can then drive the first ambient-field motor according to the first composite commutation angle.

In particular, during operation of the robotic system 200 within the bore of the MRI machine, each ambient-field motor experiences a local magnetic field vector that depends on: pose of the ambient-field motor within the bore; and operational state of the MRI machine (e.g., imaging versus non-imaging). The electrical commutation offset for an ambient-field motor varies with: pose of the ambient-field motor; and operational state of the MRI machine. Electrical commutation of an ambient-field motor depends on two distinct angle components, including: a mechanical commutation angle derived from a mechanical rotor position; and an electrical commutation offset derived from the magnetic field vector proximal the ambient-field motor and the pose of the ambient-field motor. If the electrical commutation offset is incorrect for a given time interval: motor current is phased away from a torque-producing direction relative to the local magnetic field vector of the ambient-field motor; torque output per current input to the ambient-field motor decreases; temperature of the ambient-field motor may increase; the ambient-field motor may generate more electromagnetic noise that may affect imaging quality of the MRI machine; and the transient response of the corresponding joint (e.g., overshoot, damping, settling time) may become less predictable and less controllable and may couple into other joints within the robotic arm.

### 7.1 Composite Commutation Angle Derivation

As described above, each joint can include a drive unit. Each drive unit can include: an ambient-field motor and a gearbox (e.g., to form a gearhead ambient-field motor); a rotor encoder coupled to the rotor of the ambient-field motor that outputs absolute mechanical rotor position and/or changes in mechanical rotor position; and a joint encoder that outputs an absolute joint position, changes in joint position, and/or indication that the joint occupies a reference (or “zero,”, “home”) position. For example, the rotor and joint encoders can include absolute position encoders. In another example: the rotor encoder includes an absolute position encoder; the joint encoder includes a limit switch that indicates when the joint occupies a reference position; and the ambient-field motor or the motion control interface calculates an absolute position of the joint based on rotor position data received from the rotor encoder since the joint occupied the reference position (and a gear ratio of the gearbox on the ambient-field motor).

Thus, for each joint of the robotic arm, the motion control interface 240 can receive or access: joint positions from joint encoders in the set of joints; and mechanical rotor positions from rotor encoders in the set of joints, such as at a frequency of 100 Hz (i.e., once per 10-millisecond time slot).

The motion control interface 240 can also access an MRI table position from a table encoder coupled to a table of the MRI machine on which the robotic arm is installed.

The motion control interface 240 can also access a kinematic model that relates joint positions to three-dimensional poses of the ambient-field motors. For example, the kinematic model can include a wireframe model or set of functions that outputs a three-dimensional pose (i.e., a position and orientation in six degrees of freedom) of each joint in the robotic arm—relative to a coordinate system of the robotic arm, the table, or the MRI machine—based on the joint position of each joint and the table position and known lengths of each segment of the robotic arm.

Then, for a particular ambient-field motor arranged within a particular joint in the robotic arm, the motion control interface 240: derives a current pose of the ambient-field motor within the bore of the MRI machine based on the current joint positions of the robotic arm and the kinematic model; calculates a current magnetic field vector proximal the ambient-field motor based on the pose and the magnetic field model (e.g., from memory); projects the magnetic field vector onto a plane orthogonal to a rotor rotation axis of the ambient-field motor; accesses a reference vector—such as established during initialization of the ambient-field motor—defined within this plane and fixed relative to a housing of the ambient-field motor; and calculates an electrical commutation offset for the ambient-field motor based on an angular difference between this projected magnetic field vector and the reference vector.

The motion control interface 240 also: calculates a mechanical commutation angle for the ambient-field motor based on the mechanical rotor position; combines the mechanical commutation angle and the electrical commutation offset to calculate a composite commutation angle for the ambient-field motor; and transmits the composite commutation angle (or an equivalent phased command) to a motor driver, such as arranged within the ambient-field motor or arranged outside of the MRI machine. The motor driver then supplies phased current to the coil windings of the ambient-field motor according to the composite commutation angle during a next time slot to drive articulation of the corresponding joint.

### 7.2 Commutation Updates During Motion

As described above, the correct composite commutation angle is derived from a combination (e.g., sum) of the electrical commutation offset and the mechanical commutation angle, wherein the electrical commutation offset of an ambient-field motor changes when the magnetic field exposed to the ambient-field motor changes, and wherein the mechanical commutation angle of the ambient-field motor changes only when the rotor within the ambient-field motor rotates.

Therefore, the correct composite commutation angle of a particular ambient-field motor may change between two time slots if: the rotor rotates in the ambient-field motor; an upstream joint was actuated or otherwise changed position between the prior time slot and the current time slot; a downstream joint changed motion with sufficient jerk or acceleration to shift the position of the particular ambient-field motor between the prior time slot and the current time slot; the table of the MRI machine moved between the prior time slot and the current time slot; and/or the operational state of the MRI machine changed between the prior time slot and the current time slot.

Therefore, the motion control interface 240 can repeat the foregoing processes over time (e.g., for each time slot, or once per ten milliseconds) to recalculate a composite commutation angle for the ambient-field motor even if the ambient-field motor itself is braked or otherwise was not actuated between the previous time slot and the current time slot.

For example, the motion control interface 240 can: access a first joint position of the first joint at the first time; access a second joint position of a second joint, in the set of joints and interposed between the first joint and a base of the robotic system 200, at the first time; calculate a first mechanical commutation angle based on the first mechanical rotor position; calculate a first electrical commutation offset based on the first magnetic field offset; and calculate the first composite commutation angle based on a combination of the first mechanical commutation angle and the first electrical commutation offset. The motion control interface 240 can then: access a third joint position of the first joint at a second time, the third joint position equal to the first joint position; access a fourth joint position of the second joint at the second time, the fourth joint position different from the second joint position; access a second mechanical rotor position of the first rotor at the second time, the second mechanical rotor position equal to the first mechanical rotor position; derive a second pose of the first ambient-field motor within the MRI machine at the second time based on the fourth joint position, the second pose different from the first pose; characterize a second magnetic field offset between the first ambient-field motor and the magnetic field of the MRI machine proximal the first ambient-field motor at the second time based on the second pose of the first ambient-field motor, the second magnetic field offset different from the first magnetic field offset; calculate a second mechanical commutation angle based on the second mechanical rotor position, the second mechanical commutation angle equal to the first mechanical commutation angle; calculate a second electrical commutation offset based on the second magnetic field offset, the second electrical commutation offset different from the first electrical commutation offset; and calculate a second composite commutation angle based on a second combination of the second mechanical commutation angle and the second electrical commutation offset, the second composite commutation angle different from the first composite commutation angle. The motor driver can then drive the first ambient-field motor according to the second composite commutation angle following the second time.

### 7.3 Magnetic Field Selection by MRI Operational State

In one variation in which the robotic system generates, stores, and/or accesses multiple field maps associated with different operational (or imaging) modes of the MRI machine, the motion control interface 240: polls a current MRI operational state from MRI controller or otherwise receives operational state changes from the MRI machine; selects a magnetic field map (e.g., from memory) that corresponds to operational state of the MRI machine; and calculates the electrical commutation offset, composite commutation angle, torque constant of each ambient-field motor, and/or closed-loop control coefficients for each ambient-field motor for the current time slot based on this field map.

In one implementation, the motion control interface 240: accesses a first imaging-state indicator indicating a non-imaging state of the MRI machine during the first time; accesses a first magnetic field model representing magnetic field vectors within the bore of the MRI machine according to the first imaging-state indicator; and calculates the first magnetic field offset between the first ambient-field motor and a first magnetic field vector of the magnetic field of the MRI machine proximal the first ambient-field motor at the first time based on the first pose of the first ambient-field motor and the first magnetic field model. Later, the motion control interface 240: accesses a second set of joint positions of the set of joints at a second time; accesses a second mechanical rotor position of the first rotor at the second time; derives a second pose of the first ambient-field motor within the MRI machine at the second time based on the second set of joint positions; accesses a second imaging-state indicator indicating an imaging state of the MRI machine during the second time; accesses a second magnetic field model—different from the first magnetic field model—representing magnetic field vectors within the bore of the MRI machine according to the second imaging-state indicator; calculates a second magnetic field offset between the first ambient-field motor and a second magnetic field vector of the magnetic field of the MRI machine proximal the first ambient-field motor at the second time based on the second pose of the first ambient-field motor and the second magnetic field model; and calculates a second composite commutation angle for the first ambient-field motor based on the second mechanical rotor position and the second magnetic field offset.

Therefore, the motion control interface 240 can: store multiple magnetic field models, each corresponding to a different operational state of the MRI machine; receive an MRI operational-state indicator from the MRI machine; and select and implement a magnetic field model that corresponds to the current operational state of the MRI machine, thereby generating composite commutation angles to reflect and compensate for different magnetic fields generated by the MRI machine when executing different imaging routines.

### 7.4 Composite Commutation Angle Derivation at Ambient-Field Motor

Additionally or alternatively, each ambient-field motor (or drive unit, joint) can implement the foregoing methods and techniques to locally derive its composite commutation angle during each time slot. In particular, in this variation, each ambient-field motor (or drive unit, joint) can: report its rotor and joint positions during each time slot to each other ambient-field motor in the robotic arm, such as over local wired communication protocol; store local copies of the magnetic field model of the MRI machine and the kinematic model of the robotic arm; and locally derive its composite commutation angle during each time slot.

In one example, a first ambient-field motor arranged within a first joint in the robotic arm can: read a first joint position of the first joint at the current time from a first joint encoder within the first ambient-field motor; receive a second joint position, of a second joint in the set of joints of the robotic system 200 at the current time, from a second ambient-field motor arranged within a second joint in the robotic arm via a wired connection between the first ambient-field motor and the second ambient-field motor; access the kinematic model from a first local memory within the first ambient-field motor; derive a first pose of the first ambient-field motor within the MRI machine at the current time based on the first joint position, the second joint position, and the kinematic model; access the three-dimensional magnetic field model from the first local memory; calculate a first magnetic field offset (e.g., an angular offset) between the first ambient-field motor and a magnetic field vector of the magnetic field of the MRI machine proximal the first ambient-field motor at the current time based on the first pose of the first ambient-field motor and the three-dimensional magnetic field model; calculate a first electrical commutation offset at the current time based on the first magnetic field offset; and calculate a first composite commutation angle for the first ambient-field motor at the current time accordingly. The first ambient-field motor can then: locally implement this first composite commutation angle via a motor driver within the first ambient-field motor; or transmit the first composite commutation angle to an external motor driver arranged outside of the MRI machine.

Therefore, in this variation, the ambient-field motor can: include local memory; store a magnetic field model of the MRI machine and a kinematic model of the robotic arm in the local memory; include an internal motor controller; receive joint positions from all other ambient-field motors in the robotic arm; and locally implement methods and techniques described above to calculate its electrical offset and derive its composite commutation angle.

### 7.5 Electrical Commutation Offset From Internal Sensing

Additionally or alternatively, each ambient-field motor (or drive unit, joint) can include a magnetic field sensor configured to directly sense magnetic field magnitude and orientation proximal its rotor, as shown in FIG. 6.

For example, each ambient-field motor can: include a three-axis Hall effect sensor arranged immediately adjacent the rotor within the ambient-field motor; directly sample the three-axis Hall effect sensor during a time slot; calculate a three-axis magnetic field vector in a coordinate frame of the three-axis Hall effect sensor based on magnetic field magnitudes received from the three-axis Hall effect sensor; transform the three-axis magnetic field vector from the sensor coordinate frame into a rotor coordinate frame based on a known offset between the three-axis Hall effect sensor and the rotor; and calculate its electrical commutation offset for the current time slot based on the magnitude and orientation of the magnetic field vector in the rotor coordinate frame.

In another example, each ambient-field motor can: include a two-axis Hall effect sensor arranged immediately adjacent the rotor within the ambient-field motor and with each axis of the two-axis Hall effect sensor arranged perpendicular to the rotation axis of the ambient-field motor; directly sample the two-axis Hall effect sensor during a time slot; calculate a magnitude of the magnetic field within a plane normal to this rotation axis based on outputs of the two-axis Hall effect sensor; and transform this magnitude into an electrical commutation offset for the current time slot.

In these examples, the ambient-field motor can then: calculate its mechanical commutation angle for the current time slot based on a rotor position read from the rotor encoder during the current time slot; combine its electrical commutation offset and mechanical commutation angle to calculate its composite commutation angle for the current time slot; and report this composite commutation angle to its internal motor driver, the motion control interface 240, or an external motor driver.

### 7.6 Refence Electrical Commutation Offset From Motor Performance

Additionally or alternatively, the motion control interface 240 (or an ambient-field motor) can estimate the electrical commutation offset of an ambient-field motor based on back-EMF characteristics of the ambient-field motor when energized or moved within the magnetic field of the MRI machine. In particular, as the rotor of an ambient-field motor moves relative to the magnetic field of the MRI machine, the coil windings generate back-EMF signals characterized by phase and polarity that depend on angular offset between the rotor and the local magnetic field vector proximal the rotor. Thus, during rotation, micro-motion, probing pulses, or induced motion from other joints, the motion control interface 240 can record back-EMF characteristics of the ambient-field motor and estimate an electrical commutation offset based on these back-EMF characteristics.

In one implementation, the motion control interface 240 (or the ambient-field motor) can: monitor back-EMF signals from one or more coil windings in the ambient-field motor during rotation of its rotor, during micro-motion of the rotor, or when the corresponding joint is moved within the bore of the MRI machine due to actuation of another joint in the robotic arm; detect phase relationships or zero-crossing behavior in these back-EMF signals; and derive a performance-based electrical commutation offset-for the ambient-field motor for the current time-that a) aligns phase timing of drive currents to the ambient-field motor with a torque-producing direction indicated by the observed back-EMF, b) corrects phase timing of drive currents based on a phase relationship between the back-EMF and rotor motion, and/or c) compensates for misalignment between a commanded commutation phase and an actual torque-producing phase indicated by the back-EMF.

In this variation, the motion control interface can additionally or alternatively implement similar methods and techniques to derive the composite commutation angle directly from these back-EMF signals.

### 7.7 Redundant Electrical Angle Derivation+Error Handling

In one variation, the motion control interface 240 (and/or the ambient-field motors): implements multiple techniques described above to derive multiple electrical commutation offset per time slot; compares concurrent electrical commutation offsets; detects error or failure in the robotic system 200 responsive to differences between concurrent electrical commutation offsets; and reduces motion limits for each joint, ceases operation of each joint, and/or brakes each joint responsive to such errors or failures.

In one implementation, the motion control interface 240 implements methods and techniques described above to: calculate a kinematics-based electrical commutation offset for an ambient-field motor for the current time slot; and to calculate a performance-based electrical commutation offset for the ambient-field motor for the current time slot. Then, if these two electrical commutation offsets differ by more than a threshold difference (e.g., 10%), the motion control interface 240 can: broadcast an alarm; or increase a minimum difference between an estimated torque output capacity of the ambient-field motor and a torque demand to the ambient-field motor at any time thereafter until this difference falls below the threshold difference.

For example, the motion control interface 240 can: access a set of joint positions of the set of joints at a current time; access a mechanical rotor position of the rotor at the current time; derive a pose of the ambient-field motor within the MRI machine at the current time based on the set of joint positions; calculate a magnetic field offset between the ambient-field motor and a magnetic field vector of the magnetic field of the MRI machine proximal the ambient-field motor at the current time based on the pose of the ambient-field motor; calculate a composite commutation angle for the ambient-field motor based on the mechanical rotor position and the magnetic field offset; access a back-electromotive force of the ambient-field motor at the current time; and derive a reference commutation offset of the ambient-field motor at the current time based on the back-electromotive force. If the composite commutation angle differs from the reference commutation offset by more than a threshold difference (e.g., 2%), the motion control interface 240 can generate an alarm.

Additionally or alternatively, the motion control interface 240 and an ambient-field motor can similarly cooperate to: independently derive kinematics-based and sensor-based electrical commutation offsets for the ambient-field motor for a time slot; and generate an alarm or adjust limits of the robotic system 200 responsive to differences between these electrical commutation offsets.

Additionally or alternatively, the motion control interface 240 and/or the ambient-field motor can derive and compare magnetic field offsets between the ambient-field motor and the magnetic field of the MRI machine (i.e., rather than electrical commutation offsets).

### 7.8 Magnetic Field Model Validation

In another variation, the robotic arm includes a reference magnetic field sensor, such as a single-axis, two-axis, or three-axis Hall effect sensor arranged in a distal joint of the robotic arm or in an end effector arranged on the robotic arm.

In this variation, the motion control interface 240 can: read a magnetic field magnitude and orientation from the magnetic field sensor during a current time slot; and calculate an actual magnetic field vector for the current time period based on this magnitude and orientation; access a set of joint positions of the set of joints within the robotic system 200 in the current time slot; derive a pose of the magnetic field sensor within the MRI machine in the current time slot based on the set of joint positions and the kinematics model of the robotic system 200, which represents a known position of the magnetic field sensor on the robotic arm; and calculate a reference magnetic field vector at the magnetic field sensor based on the pose of the magnetic field sensor and the magnetic field model of the MRI machine. Then, if the actual magnetic field vector and the reference magnetic field vector differ by more than a threshold difference in magnitude or direction (e.g., 5%, 3°), the motion control interface 240 can implement methods and techniques described above to generate an alarm or adjust limits of the robotic system 200.

For example, the motion control interface 240 can: access an actual magnetic field vector detected by the magnetic field sensor at the current time; and derive a predicted magnetic field vector—at a location of the magnetic field sensor on the robotic system 200—at the first time based on current joint positions of joints in the robotic arm and the three-dimensional magnetic field model associated with the MRI machine. Then, if the actual magnetic field vector differs from the predicted magnetic field vector by more than a threshold difference (e.g., 1%, 2%, 10%), the motion control interface 240 can reduce motion limits of the set of joints, as described below.

### 8. Varying Torque Constant and Torque Limit Controls

As shown in FIG. 4, each ambient-field motor exhibits a pose-dependent torque constant within the MRI machine. In particular, the torque constant of an ambient-field motor during the current time slot may vary with: magnitude and orientation of the magnetic field proximal the ambient-field motor during the current time slot, which may be a function of the operational state of the MRI machine; and orientation of the rotor axis of the ambient-field motor relative to the magnetic field during the current time slot. Therefore, the torque output of the ambient-field motor per unit current supplied to the ambient-field motor may vary continuously during motion of the robotic arm.

However, the ambient-field motor may be current-limited, such as due to motor internal resistance, coil wire diameter, and/or electromagnetic noise limits within the MRI machine. Therefore, the robotic system 200 may be unable to recover from reduction in torque constant of an ambient-field motor and corresponding reduction in torque output capacity of the ambient-field motor occupying certain poses—when torque demand on the ambient-field motor is high—by merely supplying more current to the ambient-field motor. In particular, torque demand on the ambient-field motor may vary with: commanded motion of the corresponding joint; motion of upstream and downstream joints, which may produce coupled impulses into the corresponding joint and thus into the ambient-field motor; weight and inertia of downstream joints, arm segments, and an end effector located on the robotic arm; and/or loading of the end effector when in contact with a patient.

Thus, the torque output capacity of the ambient-field motor and the torque demand on the ambient-field motor may vary independently and simultaneously (i.e., in both the same and different directions over time).

Accordingly, the robotic system 200 can evaluate a torque margin (i.e., a difference between concurrent torque output capacity and torque demand) of the ambient-field motor based on: a derived torque constant of the ambient-field motor; a fixed or known current limit of the ambient-field motor; and/or an estimated current or future torque demand on the ambient-field motor arising from commanded pose holds and motion of the robotic arm. The motion control interface 240 can then coordinate motion of all joints in the robotic arm in order to preserve sufficient torque margin at this particular ambient-field motor during operation of the robotic arm.

### 8.1 Torque Output Capacities

In one implementation, the motion control interface 240 continuously evaluates a torque margin of an ambient-field motor based on: a pose-dependent torque constant of the ambient-field motor; a current limit of the ambient-field motor; and an estimated torque demand at the ambient-field motor. The motion control interface 240 then constrains torque commanded from the ambient-field motor in order to: prevent this commanded torque from exceeding the torque output capacity of the ambient-field motor (within a tolerance or safety margin); and preserve control authority of the ambient-field motor into the next time slot. Accordingly, the motion control interface 240 can: implement torque limiting for the ambient-field motor torque before reaching torque saturation in order to: prevent loss of positional control at the corresponding joint; prevent uncontrolled current rise at the ambient-field motor; and/or maintain predictable behavior of the corresponding joint—whether in motion or holding position—through the next time slot.

Therefore, the motion control interface 240 can update an estimate of torque output capacity of the ambient-field motor dynamically—and limit torque commanded from the ambient-field motor accordingly—as pose of the ambient-field motor changes within the bore of the MRI machine.

For example and as described above, the motion control interface 240 can: access a planned motion of the robotic system 200 following the first time, the planned motion defining a traversal of the first joint from a first position to a second position; predict a first set of joint positions of the set of joints at a future time following the first time based on the planned motion; derive a first pose of a first ambient-field motor within the MRI machine at the future time based on the first set of joint positions, the first ambient-field motor arranged within a first joint in the set of joints of the robotic system 200; characterize a first magnetic field offset between the first ambient-field motor and the magnetic field of the MRI machine proximal the first ambient-field motor at the future time based on the first pose of the first ambient-field motor; calculate a future torque constant of the first ambient-field motor at the future time based on the first magnetic field offset; calculate a future torque output capacity of the first ambient-field motor at the future time based on the future torque constant and the current limit of the first ambient-field motor; and estimate a future torque demand of the first ambient-field motor at the future time based on the kinematic model of the robotic system 200 and the planned motion. Accordingly, in response to the future torque demand approaching the future torque output capacity of the first ambient-field motor, the motion control interface 240 can reduce a torque output commanded of the first ambient-field motor.

Therefore, in this implementation, the motion control interface 240 can: implement the torque output capacity of the ambient-field motor as a pose-dependent constraint rather than a fixed property of the ambient-field motor; proactively limit commanded torque output of the ambient-field motor before current saturation or torque collapse occurs at the ambient-field motor; and avoid initiating or continuing robotic arm maneuvers in which the torque demanded from the ambient-field motor exceeds the physically-achievable current-limited torque output capacity of the ambient-field motor givens its current and upcoming magnetic field exposure. The motion control interface 240 can thus: preserve control authority of the corresponding joint, even across a range of joint poses in which magnetic field coupling to the ambient-field motor is significantly degraded (e.g., reduced by 80% from a maximum magnetic field coupling); and bound motor current, control thermal load, and limit electromagnetic emissions from the ambient-field motor while the robotic arm operates within the MRI machine.

### 8.2 Acceleration Limits

In another implementation, the motion control interface 240 implements pose-dependent acceleration limits for an ambient-field motor. In particular, the inertial torque output by an ambient-field motor to achieve a commanded acceleration at the corresponding joint may depend on inertia of the robotic arm, geometry and current pose of the robotic arm, and the position of the joint within the robotic arm.

However, the torque constant of the ambient-field motor varies with its pose within the magnetic field of the MRI machine, and the achievable current-limited output torque capacity of the ambient-field motor changes as the ambient-field motor moves within the bore of the MRI machine. Therefore, the motion control interface 240 can set acceleration limits of the ambient-field motor in order to preserve control authority of the joint through the next time slot based on inertia of the robotic arm, geometry and current pose of the robotic arm, and the position of the joint within the robotic arm

For example, the motion control interface 240 can: access a first set of joint positions of a set of joints, within a robotic system 200 occupying a bore of an MRI machine, at a first time; access a first mechanical rotor position of a first rotor, in a first ambient-field motor arranged within a first joint in the set of joints of the robotic system 200, at the first time; derive a first pose of the first ambient-field motor within the MRI machine at the first time based on the first set of joint positions; characterize a first magnetic field offset between the first ambient-field motor and a magnetic field of the MRI machine proximal the first ambient-field motor at the first time based on the first pose of the first ambient-field motor; and calculate a first composite commutation angle for the first ambient-field motor based on the first mechanical rotor position and the first magnetic field offset. The motor driver can then drive the first ambient-field motor according to the first composite commutation angle to accelerate rotation of the first joint at a first acceleration rate. The motion control interface 240 can then: access a second set of joint positions of the set of joints at a second time succeeding the first time; derive a second pose of the first ambient-field motor within the MRI machine at the second time based on the second set of joint positions; characterize a second magnetic field offset between the first ambient-field motor and the magnetic field of the MRI machine proximal the first ambient-field motor at the second time based on the second pose of the first ambient-field motor; characterize a torque constant of the first ambient-field motor at the second time based on the second magnetic field offset; calculate a torque output capacity of the first ambient-field motor at the second time based on the torque constant of the first ambient-field motor; access a kinematic model that relates joint positions of the robotic system 200 to three-dimensional positions of the set of joints; and estimate an inertial torque demand of the first ambient-field motor at the second time based on the kinematic model, the second set of joint positions, and the first acceleration rate. Then, in response to the inertial torque demand approaching the torque output capacity of the first ambient-field motor, the motion control interface 240 can reduce the first acceleration rate of the first joint following the second time.

In a similar implementation, the motion control interface 240 can implement joint acceleration as a torque-producing command subject to a pose-dependent torque output capacity. Accordingly, the motion control interface 240 can derive an acceleration-dependent torque demand for the ambient-field motor based on: joint inertia coupled into the ambient-field motor via the corresponding gearbox; real-time geometry of the robotic arm (e.g., based on the kinematic model and current joint positions); and a commanded acceleration rate of joint within the robotic arm. The motion control interface 240 can then: compare this acceleration-dependent torque demand to the current-limited torque output capacity of the ambient-field motor at the current time; and revise acceleration limits for the ambient-field motor and/or modify acceleration commands issued to the ambient-field motor in order to maintain the acceleration-dependent torque demand below the current-limited torque output capacity of the ambient-field motor.

Therefore, in this implementation, the motion control interface 240 can: bound acceleration commands for the ambient-field motor to values that remain physically realizable for the ambient-field motor given its current pose within the MRI machine; prevent loss of acceleration tracking caused by torque saturation of the ambient-field motor as its magnetic field coupling weakens; maintain predictable rise times and damping of the corresponding joint despite pose-dependent variations in torque constant of the ambient-field motor; and reduce transient current spikes and avoid uncontrolled motion, oscillation, or delay in convergence on a target joint position resulting from over-aggressive acceleration commands issued to the ambient-field motor.

### 8.3 Joint Actuation Envelope

In another implementation, the motion control interface 240 can prevent loss of control authority at a second ambient-field motor by narrowing the motion envelope of a first (e.g., upstream) ambient-field motor, thereby preventing the first ambient-field motor from moving the second ambient-field motor too close to the bore opening of the MRI machine or rotating the second ambient-field motor to a pose that yields insufficient coupling to the magnetic field of the MRI machine to preserve torque margin at the ambient-field motor.

More specifically, motion of the first ambient-field motor within its motion envelope may move the second ambient-field motor—downstream of the first ambient-field motor—near the bore opening of the MRI machine where the density and orientation of the magnetic field of the MRI machine changes, thereby reducing coupling to the second ambient-field motor and reducing the torque constant of the second ambient-field motor—even if the angular position of the second application group within the bore is unchanged. The motion control interface 240 can predict such upcoming losses in torque margin at the second end effector and preemptively narrow the motion envelope of the first joint during subsequent time slots in order to avoid these losses in torque margin at the second ambient-field motor.

For example, the motion control interface 240 can: access a first set of joint positions of a set of joints, within a robotic system 200 occupying a bore of an MRI machine, at a first time; access a first mechanical rotor position of a first rotor, in a first ambient-field motor arranged within a first joint in the set of joints of the robotic system 200, at the first time; derive a first pose of the first ambient-field motor within the MRI machine at the first time based on the first set of joint positions; characterize a first magnetic field offset between the first ambient-field motor and a magnetic field of the MRI machine proximal the first ambient-field motor at the first time based on the first pose of the first ambient-field motor; and calculate a first composite commutation angle for the first ambient-field motor based on the first mechanical rotor position and the first magnetic field offset. The motor driver can then drive the first ambient-field motor according to the first composite commutation angle to rotate the first joint from a first joint position to a second joint position within a first position envelope of the first ambient-field motor. The motion control interface 240 can then: access a second set of joint positions of the set of joints at a second time; derive a second pose of a second ambient-field motor, arranged within a second joint in the set of joints of the robotic system 200, within the MRI machine at the second time based on the second set of joint positions; characterize a second magnetic field offset between the second ambient-field motor and the magnetic field of the MRI machine proximal the second ambient-field motor at the second time based on the second pose of the second ambient-field motor; characterize a torque constant of the second ambient-field motor at the second time based on the second magnetic field offset; calculate a torque output capacity of the second ambient-field motor at the second time based on the torque constant and a current limit of the second ambient-field motor; and estimate a current torque demand of the second ambient-field motor during the second time based on a kinematic model of the robotic system 200 and motion of the robotic system 200 from the first time to the second time. Then, in response to the current torque demand approaching the torque output capacity of the second ambient-field motor, the motion control interface 240 can narrow the first position envelope of the first ambient-field motor beyond the second joint position.

Therefore, in this example, the motion control interface 240 can set a width of the motion envelope in which the first ambient-field motor is permitted to operate or otherwise narrow this motion envelope, such as proportional to the torque margin of the second ambient-field motor. For example, the motion control interface 240 can enable the robotic system 200 to drive the first ambient-field motor to a pose that positions the second ambient-field motor near the bore opening of the MRI machine when the torque demand to the second ambient-field motor is low; and vice versa. More specifically, the motion control interface 240 can reduce the motion envelope of the first ambient-field motor in order to avoid loss of control authority at the second ambient-field motor as a function of torque margin of the second ambient-field motor.

Therefore, the motion control interface 240 can override mechanical rotation limits of a joint with narrower rotation limits based on predicted reduction in torque margin at an ambient-field motor in a second, downstream joint in the robotic arm in order to preserve control authority in the second, downstream joint.

### 8.4 Joint Actuation Speed

In another implementation, the motion control interface 240: implements joint speed commands as a shared system resource constrained by pose-dependent torque authority; and sets limits on speeds commanded of a first joint in light of dynamic torque demands-induced by motion of the first joint-at other joints in the robotic arm in order to preserve control authority across the entire robotic arm during operation. More specifically, the motion control interface 240 can constrain speed commands issued to a first ambient-field motor in a first joint such that no other ambient-field motor in any other joint in the robotic arm is forced into a torque deficit while attempting to maintain its commanded motion or holding state due to motion of the first joint.

In particular, inertial and coupling torques experienced by a second joint may be proportional to the actuation speed of a first joint in the robotic arm; and the torque constant of a second ambient-field motor in the second joint may vary with pose of the robotic arm and the operating state of the MRI machine. Accordingly, the second motor may exhibit a sufficient current-limited torque output capacity to maintain its commanded position, speed, or acceleration, in one pose but may lose torque authority to maintain this commanded position, speed, or acceleration in another pose. Therefore, the motion control interface 240 can avoid loss of positional control of the end effector by reducing speed limits of both upstream and downstream joints in order to preserve control authority of a particular joint containing an ambient-field motor currently exhibiting least torque margin during a time slot.

For example, the motion control interface 240 can: access a first set of joint positions of a set of joints, within a robotic system 200 occupying a bore of an MRI machine, at a first time; access a first mechanical rotor position of a first rotor, in a first ambient-field motor arranged within a first joint in the set of joints of the robotic system 200, at the first time; derive a first pose of the first ambient-field motor within the MRI machine at the first time based on the first set of joint positions; characterize a first magnetic field offset between the first ambient-field motor and a magnetic field of the MRI machine proximal the first ambient-field motor at the first time based on the first pose of the first ambient-field motor; and calculate a first composite commutation angle for the first ambient-field motor based on the first mechanical rotor position and the first magnetic field offset.

The motor driver can then drive the first ambient-field motor according to the first composite commutation angle to rotate the first joint at a first speed. The motion control interface 240 can then: access a second set of joint positions of the set of joints at a second time; derive a second pose of a second ambient-field motor, arranged within a second joint in the set of joints of the robotic system 200, within the MRI machine at the second time based on the second set of joint positions; characterize a second magnetic field offset between the second ambient-field motor and the magnetic field of the MRI machine proximal the second ambient-field motor at the second time based on the second pose of the second ambient-field motor; calculate a torque constant of the second ambient-field motor at the second time based on the second magnetic field offset; calculate a torque output capacity of the second ambient-field motor at the second time based on the torque constant and a current limit of the second ambient-field motor; and estimate a current torque demand of the second ambient-field motor during the second time based on a kinematic model of the robotic system 200 and motion of the robotic system 200 from the first time to the second time. Then, in response to the current torque demand approaching the torque output capacity of the second ambient-field motor, the motion control interface 240 can reduce speeds of joints in the robotic system 200 following the second time.

Therefore, in this implementation, the motion control interface 240 can: dynamically reduce joint speeds as ambient-field motors move into poses that yield reduced magnetic coupling and/or reduce torque margin; prevent loss of holding force or speed tracking at joints experiencing diminished torque authority or narrow torque margin; avoid injecting impulsive loads into these joints by constraining motion of other joints in the robotic arm; maintain smooth, monotonic progression of the end effector along a commanded trajectory; and thus ensure that reductions in speed of joints in the arm occur as controlled adaptations rather than as uncontrolled stalls or oscillations.

### 8.5 Path Modification for Control Authority Preservation

In another implementation, the motion control interface 240 (or other computer system, motor driver, system-level controller, or surgeon interface): implements a planned path to drive an end effector on the robotic arm to a final target pose as a provisional trajectory subject to modification based on predicted electromechanical feasibility of the planned path; evaluates future torque authority of ambient-field motors within the robotic arm as the robotic arm traverses the planned path; identifies a segment of the planned path in which the pose of a particular ambient-field motor may reduce the torque margin of the ambient-field motor below a threshold (e.g., safety) margin; and preemptively modifies the planned path before or during execution in order to drive the end effector to the same final target pose while increasing or otherwise preserving sufficient torque margin of the particular ambient-field motor (i.e., rather than react only after the particular ambient-field motor reaches such torque limits during execution of the original planned path).

For example, the motion control interface 240 can: access a planned motion of the robotic system 200 following the first time, the planned motion defining traversal of the first joint from a first joint position to a second joint position to drive an end effector on the robotic system 200 along a first path from a first end effector position to a second end effector position within the bore of the MRI machine; predict a future pose of a second ambient-field motor, arranged within a second joint in the set of joints of the robotic system 200, based on the planned motion; characterize a future magnetic field offset between the second ambient-field motor and the magnetic field of the MRI machine, during execution of the planned motion by the robotic system 200, based on the future pose; calculate a future torque output capacity of the second ambient-field motor, during execution of the planned motion by the robotic system 200, based on the future magnetic field offset and a current limit of the second ambient-field motor; access a kinematic model that relates joint positions of the robotic system 200 to three-dimensional positions of the set of joints; and estimate a future torque demand of the second ambient-field motor, during execution of the planned motion by the robotic system 200, based on the kinematic model of the robotic system 200 and the planned motion. Then, in response to the future torque demand of the second ambient-field motor approaching the future torque output capacity of the second ambient-field motor, the motion control interface 240 can modify the planned motion to: define traversal of the first joint from the first joint position to a third joint position to drive the end effector along a second path, different from the first path, from the first end effector position to the second end effector position; and increase a future margin between the future torque output capacity and the future torque demand of the second ambient-field motor during execution of the planned motion by the robotic system 200.

In another implementation, when the robotic system 200 (e.g., the end effector) is in contact with a patient, the motion control interface 240 (or other computer system, motor driver, system-level controller, or surgeon interface) can: access a planned path of the end effector from a current pose to a target pose within the bore of the MRI machine; access a planned path speed of the end effector from the current pose to the target pose; define an initial sequence of poses of the robotic arm to traverse the end effector along this planned path based on the kinematic model of the robotic system 200; predict (or “simulate”) future torque demands at each ambient-field motor during execution of the initial sequence of poses based on the kinematic model of the robotic system 200 and the planned path speed; predict future torque constants and/or torque output capacities of each ambient-field motor during execution of the initial sequence of poses based on the magnetic field model of the MRI machine; and predict future torque margins of each ambient-field motor during execution of the initial sequence of poses based on these future torque output capacities and corresponding future torque demands.

Then, in response to a future torque margin of a particular ambient-field motor falling below a threshold (e.g., safety) margin, the motion control interface 240 can modify the sequence of poses—while preserving the planned path of the end effector between the current pose and the target pose—and repeat the foregoing process to validate torque margins of the ambient-field motors throughout execution of the revised sequence of poses. The motion control interface 240 can: iteratively repeat this process until the motion control interface 240 calculates or converges on a particular sequence of poses that achieves sufficient torque margin at each joint and/or maximizes torque margin at each joint. (Furthermore, if this sequence of poses maximizes torque margin of the ambient-field motors but at least one torque margin remains less than the threshold margin, the motion control interface 240 can then selectively reduce the path speed for the sequence of poses in order to increase this margin.) The robotic system 200 can then execute this path.

Additionally or alternatively, when the end effector is not in contact with a patient, the motion control interface 240 can implement similar methods and techniques to generate a sequence of poses that: achieves sufficient torque margin at each joint and/or maximizes torque margin at each joint; preserves or maximizes a traversal speed of the end effector from the current position to the target position; and moves the end effector along a different trajectory.

Therefore, the motion control interface 240 can preemptively: modify a sequence of joint poses and/or reduce traversal speed of the end effector while preserving an end effector path to ensure sufficient torque margin at each joint while the robotic system 200 is in contact with the patient; and modify a sequence of joint poses and the end effector path while preserving or maximizing end effector traversal speed to ensure sufficient torque margin at each joint while the robotic system 200 is not in contact with the patient.

### 8.6 Torque Limit Brake Trigger

In another implementation, the motion control interface 240 can implement methods and techniques described above to track torque margin at each ambient-field motor during operation of the robotic arm. Then, if the torque margin of a particular ambient-field motor approaches the threshold margin and if the speed currently demanded of the corresponding joint is null, the motion control interface 240 can: command the ambient-field motor to stop motion of the joint; activate the brake in the joint to hold the position of the joint; continue to track the torque margin of the ambient-field motor; and then deactivate the brake in the joint once this torque margin exceeds the threshold margin.

Similarly, if the torque margin of the particular ambient-field motor approaches the threshold margin and if the speed currently demanded of the corresponding joint is not null, then the motion control interface 240 can: command the particular ambient-field motor to stop motion of the joint and activate the brake in the particular joint to hold the position of the joint; implement methods and techniques described above to modify a planned sequence of poses of other joints in the robotic arm to achieve the target path and/or target pose of the end effector now that the particular ambient-field motor is locked in its braked position; and continue to track the torque margin of the particular ambient-field motor.

Later, once this torque margin exceeds the threshold margin, the motion control interface 240 can: ramp a holding current to the particular ambient-field motor; deactivate the brake in the particular joint; and again implement methods and techniques described above to modify the planned sequence of poses of all joints in the robotic arm to achieve the target path and/or target pose of the end effector now that the particular ambient-field motor is unbraked.

Therefore, the motion control interface 240 can: detect degradation of holding authority of an ambient-field motor within a joint; and automatically trigger transfer of holding responsibility at the joint from the ambient-field motor to a mechanical brake in the joint before loss of position, oscillation, or excessive current draw at the joint.

For example, the motion control interface 240 can: access a first set of joint positions of a set of joints, within a robotic system 200 occupying a bore of an MRI machine, at a first time; access a first mechanical rotor position of a first rotor, in a first ambient-field motor arranged within a first joint in the set of joints of the robotic system 200, at the first time; derive a first pose of the first ambient-field motor within the MRI machine at the first time based on the first set of joint positions; characterize a first magnetic field offset between the first ambient-field motor and a magnetic field of the MRI machine proximal the first ambient-field motor at the first time based on the first pose of the first ambient-field motor; and calculate a first composite commutation angle for the first ambient-field motor based on the first mechanical rotor position and the first magnetic field offset. The motor driver can then drive the first ambient-field motor according to the first composite commutation angle to hold the first joint in a first joint position. The motion control interface 240 can then access a motor current amplitude of current supplied to the first ambient-field motor following the first time to hold the first joint in the first joint position. In response to the motor current amplitude approaching a current limit of the first ambient-field motor, the motion control interface 240 can: trigger a mechanical brake within the first joint to lock the first joint in the first joint position; and reduce the motor current amplitude of current supplied to the first ambient-field motor.

Therefore, the motion control interface 240 can: evaluate a holding torque demand relative to torque output capacity of an ambient-field motor; and selectively engage a mechanical brake in the corresponding joint in order to preserve joint position while reducing electrical stress, thermal load, and magnetic interference at the joint when holding torque demand approaches the torque output capacity of the ambient-field motor.

### 9. Dynamic Closed-Loop Control Coefficients

As shown in FIG. 4 during operation of the robotic system 200 within the bore of the MRI machine, the electromechanical gain of an ambient-field motor varies as a function of: orientation of the rotor axis of the ambient-field motor relative to the magnetic field of the MRI machine; and magnitude of the magnetic field proximal the ambient-field motor (which may vary with operational state of the MRI machine). Because torque constant varies with pose of the ambient-field motor (and the operational state of the MRI machine), the effective plant gain between motor current and torque at the corresponding joint may not be constant over time. If the robotic system 200 implements fixed closed-loop control coefficients for the ambient-field motor across different poses (and operational states of the MRI machine), the corresponding joint may therefore exhibit: variable overshoot as a function of pose; variable damping ratio as a function of pose; and variable time-to-target as a function of pose.

Furthermore, all joints in the robotic arm may simultaneously exhibit dissimilar dynamic responses. Such dynamic mismatches may yield: inter-joint fighting during coordinated motion; increased current demand across all joints; elevated temperatures of all ambient-field motors; MRI image quality degradation due to increased electromagnetic activity within the robotic arm; and/or increased mechanical wear of ambient-field motors and gearboxes within each joint.

Therefore, the motion control interface 240 (and/or other computer system, motor driver, system-level controller, or surgeon interface) can: characterize a torque constant of an ambient-field motor during a current time slot based on its current pose and the magnetic field model of the MRI machine; implement a stored function to calculate a set of closed-loop control coefficients (e.g., proportional gain, integral gain, and derivative gain coefficients) for the ambient-field motor during a next time slot based on the torque constant of the ambient-field motor; and then generate a motor current command for the first ambient-field motor for the next time slot based on these new set of control coefficients and a position error of the corresponding joint during the current and preceding time slots.

For example, the motion control interface 240 can transform the torque constant, joint pose, and/or magnetic field orientation of an ambient-field motor into closed-loop control coefficients for the ambient-field motor by: implementing a parametric function to derive closed-loop control coefficients for the ambient-field motor for the next time slot; selecting these closed-loop control coefficients from a lookup table stored in memory; or interpolating these closed-loop control coefficients from stored parameter sets, such as represented in a lookup table.

In one implementation, the motion control interface 240: characterizes a torque constant of the first ambient-field motor at the current time based on the first magnetic field offset; selects a first set of control coefficients assigned to the first ambient-field motor based on the torque constant, the first set of control coefficients including a first proportional gain, a first integral gain, and a first derivative gain; and generates a motor current command for the first ambient-field motor based on the first set of control coefficients and position error of the first joint at the current time. The motor driver can then drive the first ambient-field motor according to the first composite commutation angle and the motor current command.

### 8. Jitter+Slew Rate

In another variation, the motion control interface 240 (or other computer system, motor driver, system-level controller, or surgeon interface) controls position oscillation (or “jitter”) of a joint during holding or low-speed motion by regulating changes in composite commutation angle implemented for the corresponding ambient-field motor.

In particular, a joint may jitter when holding its position if: the joint is commanded to remain stationary; disturbance torques are communicated into the joint, such as due to end effector loading or inter-joint motion coupling; and/or closed-loop control coefficients implemented for the ambient-field motor on the joint are mismatched to the current pose and torque constant of the ambient-field motor.

Furthermore, jitter characteristics of the joint may vary over time due to: a dynamic torque constant that varies with pose of the joint; elastic energy storage resulting from gear compliance within the gearbox in the joint; deadband effects resulting from static friction within the joint; and/or time-varying magnetic field disturbances within the MRI machine, such as resulting from changes in operational state of the MRI machine.

Therefore, to reduce jitter in the joint over a range of poses of the joint within the bore of the MRI machine, the motion control interface 240 can: implement methods and techniques described above to maintain sufficient torque margin at the corresponding ambient-field motor; implement methods and techniques described above to selectively activate the brake in the joint when the torque margin of the ambient-field motor falls below a threshold margin; and/or limit rate of change in the electrical commutation offset calculated and implemented for the ambient-field motor across consecutive time slots.

In one implementation, the motion control interface 240: accesses a first set of joint positions of a set of joints, within a robotic system 200 occupying a bore of an MRI machine, at a first time; accesses a first mechanical rotor position of a first rotor, in a first ambient-field motor arranged within a first joint in the set of joints of the robotic system 200, at the first time; derives a first pose of the first ambient-field motor within the MRI machine at the first time based on the first set of joint positions; characterizes a first magnetic field offset between the first ambient-field motor and a magnetic field of the MRI machine proximal the first ambient-field motor at the first time based on the first pose of the first ambient-field motor; and calculates a first composite commutation angle for the first ambient-field motor based on the first mechanical rotor position and the first magnetic field offset. The motor driver then drives the first ambient-field motor according to the first composite commutation angle. The motion control interface 240 then: accesses a second set of joint positions of the set of joints at a second time succeeding the first time; accesses a second mechanical rotor position of the first rotor at the second time; derives a second pose of the first ambient-field motor within the MRI machine at the second time based on the second set of joint positions; calculates a second magnetic field offset between the first ambient-field motor and a second magnetic field vector of the magnetic field of the MRI machine proximal the first ambient-field motor at the second time based on the second pose of the first ambient-field motor; calculates a second composite commutation angle for the first ambient-field motor based on the second mechanical rotor position and the second magnetic field offset; and calculates a revised second composite commutation angle, that limits a rate of change of composite commutation angle for the first ambient-field motor across the first time and the second time to reduce position oscillation of the first joint, based on the first composite commutation angle and the second composite commutation angle.

For example, the motion control interface 240 can implement a low-pass filter to attenuate rapid changes in the composite commutation angle calculated for the ambient-field motor from the first time to the second time. Alternatively, the motion control interface 240 can implement a low-pass filter to attenuate rapid changes in the electrical commutation offset calculated for the ambient-field motor from the first time to the second time and then calculate the second composite commutation angle for the ambient-field motor at the second time based on the revised (e.g., filtered, attenuated) electrical commutation offset.

The motor driver can then drive the first ambient-field motor according to the revised second composite commutation angle.

Therefore, the motion control interface 240 can limit the commutation offset slew-rate of the ambient-field motor in order to avoid abrupt phase shifts in current delivery to the ambient-field motor, thereby reducing torque ripple and micro-oscillations in the joint and improving positional stability of the joint during static or low-speed actuation of the joint.

### 10. Simulation

As described above, the motion control interface 240 can preemptively modify motion limits, pose keypoints, brake positions, and/or closed-loop control coefficients, etc. of joints within the robotic arm during execution of a path by the robotic arm in order to preserve control authority of the robotic arm.

However, the motion control interface 240 (or other computer system, motor driver, system-level controller, or surgeon interface) can: execute the foregoing methods and techniques during simulation of operation of the robotic arm in order to derive motion limits, pose keypoints, brake positions, and/or closed-loop control coefficients, etc. of joints and ambient-field motors within the robotic arm prior to execution of the path or other planned motion by the robotic arm.

### 11. Conclusion

The systems and methods described herein can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor but any suitable dedicated hardware device can (alternatively or additionally) execute the instructions.

As a person skilled in the art will recognize from the previous detailed description and from the figures and claims, modifications and changes can be made to the embodiments of the invention without departing from the scope of this invention as defined in the following claims.

I claim:

1. A method comprising:
accessing a first set of joint positions of a set of joints, within a robotic system occupying a bore of an MRI machine, at a first time;
accessing a first mechanical rotor position of a first rotor, in a first ambient-field motor arranged within a first joint in the set of joints of the robotic system, at the first time;
deriving a first pose of the first ambient-field motor within the MRI machine at the first time based on the first set of joint positions;
characterizing a first magnetic field offset between the first ambient-field motor and a magnetic field of the MRI machine proximal the first ambient-field motor at the first time based on the first pose of the first ambient-field motor;
calculating a first composite commutation angle for the first ambient-field motor based on the first mechanical rotor position and the first magnetic field offset; and
driving the first ambient-field motor according to the first composite commutation angle.

2. The method of claim 1, wherein driving the first ambient-field motor according to the first composite commutation angle comprises:
supplying current to a set of coil windings, arranged on the first rotor of the first ambient-field motor, according to the first composite commutation angle to:
generate magnetic dipoles that magnetically couple to the magnetic field of the MRI machine;
rotate the first rotor;
drive a gearbox within the first joint; and
articulate the first joint.

3. The method of claim 1:
wherein accessing the first set of joint positions comprises, at a motion control interface arranged outside of the bore of the MRI machine:
receiving the first set of joint positions, read from a set of joint encoders arranged within the set of joints, from the set of joints;
wherein accessing the first mechanical rotor position of the first rotor comprises, at the motion control interface:
receiving the first mechanical rotor position, read from a first rotor encoder coupled to the first rotor, from the first ambient-field motor;
wherein deriving the first pose of the first ambient-field motor within the MRI machine comprises, at the motion control interface:
accessing a kinematic model that relates joint positions of the robotic system to three-dimensional positions of the set of joints; and
deriving the first pose of the first ambient-field motor within the MRI machine based on the first set of joint positions and the kinematic model;
wherein characterizing the first magnetic field offset between the first ambient-field motor and the magnetic field of the MRI machine comprises, at the motion control interface:
calculating the first magnetic field offset, comprising an angular offset, between the first ambient-field motor and a magnetic field vector of the magnetic field of the MRI machine proximal the first ambient-field motor at the first time based on the first pose of the first ambient-field motor;
wherein calculating the first composite commutation angle for the first ambient-field motor comprises, at the motion control interface:
calculating a first mechanical commutation angle based on the first mechanical rotor position;
calculating a first electrical commutation offset based on the first magnetic field offset; and
calculating the first composite commutation angle based on a combination of the first mechanical commutation angle and the first electrical commutation offset; and
wherein driving the first ambient-field motor according to the first composite commutation angle comprises, at a motor drive arranged outside of the bore of the MRI machine:
supplying current, phased according to the first composite commutation angle, to the first ambient-field motor following the first time to drive rotation of the first joint by the first ambient-field motor.

4. The method of claim [0015]:
wherein deriving the first pose of the first ambient-field motor within the MRI machine comprises:
accessing a kinematic model that relates joint positions of the robotic system to three-dimensional positions of the set of joints; and
deriving the first pose of the first ambient-field motor within the MRI machine based on the first set of joint positions and the kinematic model;
wherein characterizing the first magnetic field offset between the first ambient-field motor and the magnetic field of the MRI machine comprises:
accessing, from memory, a three-dimensional magnetic field model representing magnetic field vectors within the bore of the MRI machine; and
calculating the first magnetic field offset, comprising an angular offset, between the first ambient-field motor and a magnetic field vector of the magnetic field of the MRI machine proximal the first ambient-field motor at the first time based on the first pose of the first ambient-field motor and the three-dimensional magnetic field model; and
wherein calculating the first composite commutation angle for the first ambient-field motor comprises:
calculating a first mechanical commutation angle based on the first mechanical rotor position;
calculating a first electrical angle offset based on the first magnetic field offset; and
calculating the first composite commutation angle based on a combination of the first mechanical commutation angle and the first electrical commutation offset.

5. The method of claim 4:
wherein accessing the first set of joint positions of the set of joints comprises, at the first ambient-field motor:
reading a first joint position of the first joint at the first time from a first joint encoder within the first ambient-field motor; and
receiving a second joint position, of a second joint in the set of joints of the robotic system at the first time, from a second ambient-field motor arranged within the second joint via a wired connection between the first ambient-field motor and the second ambient-field motor;
wherein deriving the first pose of the first ambient-field motor within the MRI machine comprises, at the first ambient-field motor:
accessing the kinematic model from a first local memory within the first ambient-field motor; and
deriving the first pose of the first ambient-field motor within the MRI machine based on the first joint position, the second joint position, and the kinematic model;
wherein characterizing the first magnetic field offset between the first ambient-field motor and the magnetic field of the MRI machine comprises, at the first ambient-field motor:
accessing the three-dimensional magnetic field model from the first local memory; and
calculating the first magnetic field offset, comprising an angular offset, between the first ambient-field motor and a magnetic field vector of the magnetic field of the MRI machine proximal the first ambient-field motor at the first time based on the first pose of the first ambient-field motor and the three-dimensional magnetic field model;
wherein calculating the first composite commutation angle for the first ambient-field motor comprises calculating the first composite commutation angle for the first ambient-field motor at the first ambient-field motor; and
further comprising, at the first ambient-field motor, transmitting the first composite commutation angle to a motor driver arranged outside of the bore of the MRI machine.

6. The method of claim 4, further comprising, during a setup period prior to the first time:
navigating the robotic arm along a calibration path within the bore of the MRI machine based on a baseline magnetic field model of the MRI machine;
recording a series of magnetic field vectors based on outputs of a magnetic field sensor, arranged on the robotic system, during navigation of the robotic arm along the calibration path; and
constructing the three-dimensional magnetic field model of the MRI machine based on the series of magnetic field vectors.

7. The method of claim 6, further comprising:
accessing an actual magnetic field vector detected by the magnetic field sensor at the first time;
deriving a predicted magnetic field vector, at a location of the magnetic field sensor on the robotic system, at the first time based on the first set of joint positions and the three-dimensional magnetic field model; and
in response to the actual magnetic field vector differing from the predicted magnetic field vector by more than a threshold difference, reducing motion limits of the set of joints.

8. The method of claim [0015], further comprising:
accessing a second set of joint positions of the set of joints at a second time succeeding the first time;
accessing a second mechanical rotor position of the first rotor at the second time;
deriving a second pose of the first ambient-field motor within the MRI machine at the second time based on the second set of joint positions;
calculating a second magnetic field offset between the first ambient-field motor and a second magnetic field vector of the magnetic field of the MRI machine proximal the first ambient-field motor at the second time based on the second pose of the first ambient-field motor;
calculating a second composite commutation angle for the first ambient-field motor based on the second mechanical rotor position and the second magnetic field offset;
accessing a back-electromotive force of the first ambient-field motor at the second time;
deriving a reference commutation offset of the first ambient-field motor at the second time based on the back-electromotive force; and
in response to the second composite commutation angle differing from the reference commutation offset by more than a threshold offset difference, generating an alarm.

9. The method of claim [0015]:
wherein accessing the first set of joint positions at the first time comprises:
accessing a first joint position of the first joint at the first time; and
accessing a second joint position of a second joint, in the set of joints and interposed between the first joint and a base of the robotic system, at the first time;
wherein calculating the first composite commutation angle for the first ambient-field motor comprises:
calculating a first mechanical commutation angle based on the first mechanical rotor position;
calculating a first electrical commutation offset based on the first magnetic field offset; and
calculating the first composite commutation angle based on a combination of the first mechanical commutation angle and the first electrical commutation offset; and
further comprising:
accessing a third joint position of the first joint at a second time, the third joint position equal to the first joint position;
accessing a fourth joint position of the second joint at the second time, the fourth joint position different from the second joint position;
accessing a second mechanical rotor position of the first rotor at the second time, the second mechanical rotor position equal to the first mechanical rotor position;
deriving a second pose of the first ambient-field motor within the MRI machine at the second time based on the fourth joint position, the second pose different from the first pose;
characterizing a second magnetic field offset between the first ambient-field motor and the magnetic field of the MRI machine proximal the first ambient-field motor at the second time based on the second pose of the first ambient-field motor, the second magnetic field offset different from the first magnetic field offset;
calculating a second mechanical commutation angle based on the second mechanical rotor position, the second mechanical commutation angle equal to the first mechanical commutation angle;
calculating a second electrical commutation offset based on the second magnetic field offset, the second electrical commutation offset different from the first electrical commutation offset;
calculating a second composite commutation angle based on a second combination of the second mechanical commutation angle and the second electrical commutation offset, the second composite commutation angle different from the first composite commutation angle; and
driving the first ambient-field motor according to the second composite commutation angle following the second time.

10. The method of claim [0015]:
wherein driving the first ambient-field motor according to the first composite commutation angle comprises driving the first ambient-field motor according to the first composite commutation angle to rotate the first joint from a first joint position to a second joint position within a first position envelope of the first ambient-field motor; and
further comprising:
accessing a second set of joint positions of the set of joints at a second time;
deriving a second pose of a second ambient-field motor, arranged within a second joint in the set of joints of the robotic system, within the MRI machine at the second time based on the second set of joint positions;
characterizing a second magnetic field offset between the second ambient-field motor and the magnetic field of the MRI machine proximal the second ambient-field motor at the second time based on the second pose of the second ambient-field motor;
characterizing a torque constant of the second ambient-field motor at the second time based on the second magnetic field offset;
calculating a torque output limit of the second ambient-field motor at the second time based on the torque constant and a current limit of the second ambient-field motor;
estimating a current torque demand of the second ambient-field motor during the second time based on a kinematic model of the robotic system and motion of the robotic system from the first time to the second time; and
in response to the current torque demand approaching the torque output limit of the second ambient-field motor, narrowing the first position envelope of the first ambient-field motor beyond the second joint position.

11. The method of claim [0015]:
wherein driving the first ambient-field motor according to the first composite commutation angle comprises driving the first ambient-field motor according to the first composite commutation angle to rotate the first joint at a first speed; and
further comprising:
accessing a second set of joint positions of the set of joints at a second time;
deriving a second pose of a second ambient-field motor, arranged within a second joint in the set of joints of the robotic system, within the MRI machine at the second time based on the second set of joint positions;
characterizing a second magnetic field offset between the second ambient-field motor and the magnetic field of the MRI machine proximal the second ambient-field motor at the second time based on the second pose of the second ambient-field motor;
calculating a torque constant of the second ambient-field motor at the second time based on the second magnetic field offset;
calculating a torque output limit of the second ambient-field motor at the second time based on the torque constant and a current limit of the second ambient-field motor;
estimating a current torque demand of the second ambient-field motor during the second time based on a kinematic model of the robotic system and motion of the robotic system from the first time to the second time; and
in response to the current torque demand approaching the torque output limit of the second ambient-field motor, reducing speeds of joints in the robotic system following the second time.

12. The method of claim [0015]:
wherein driving the first ambient-field motor according to the first composite commutation angle comprises driving the first ambient-field motor according to the first composite commutation angle to apply torque to the first joint; and
further comprising:
accessing a planned motion of the robotic system following the first time, the planned motion defining a traversal of the first joint from a first position to a second position;
predicting a second set of joint positions of the set of joints at a future time following the first time based on the planned motion;
deriving a second pose of a second ambient-field motor within the MRI machine at the future time based on the second set of joint positions, the second ambient-field motor arranged within a second joint in the set of joints of the robotic system;
characterizing a second magnetic field offset between the second ambient-field motor and the magnetic field of the MRI machine proximal the second ambient-field motor at the future time based on the second pose of the second ambient-field motor;
calculating a future torque constant of the second ambient-field motor at the future time based on the second magnetic field offset;
calculating a future torque output limit of the second ambient-field motor at the future time based on the future torque constant and the current limit of the second ambient-field motor;
estimating a future torque demand of the second ambient-field motor at the future time based on the kinematic model of the robotic system and the planned motion; and
in response to the future torque demand approaching the future torque output limit of the second ambient-field motor, reducing a torque output commanded of the first ambient-field motor.

13. The method of claim [0015]:
wherein driving the first ambient-field motor according to the first composite commutation angle comprises driving the first ambient-field motor according to the first composite commutation angle to accelerate rotation of the first joint at a first acceleration rate; and
further comprising:
accessing a second set of joint positions of the set of joints at a second time succeeding the first time;
deriving a second pose of the first ambient-field motor within the MRI machine at the second time based on the second set of joint positions;
characterizing a second magnetic field offset between the first ambient-field motor and the magnetic field of the MRI machine proximal the first ambient-field motor at the second time based on the second pose of the first ambient-field motor;
characterizing a torque constant of the first ambient-field motor at the second time based on the second magnetic field offset;
calculating a torque output limit of the first ambient-field motor at the second time based on the torque constant of the first ambient-field motor;
accessing a kinematic model that relates joint positions of the robotic system to three-dimensional positions of the set of joints;
estimating an inertial torque demand of the first ambient-field motor at the second time based on the kinematic model, the second set of joint positions, and the first acceleration rate; and
in response to the inertial torque demand approaching the torque output limit of the first ambient-field motor, reducing the first acceleration rate of the first joint following the second time.

14. The method of claim [0015], further comprising:
accessing a planned motion of the robotic system following the first time, the planned motion defining traversal of the first joint from a first joint position to a second joint position to drive an end effector on the robotic system along a first path from a first end effector position to a second end effector position within the bore of the MRI machine;
predicting a future pose of a second ambient-field motor, arranged within a second joint in the set of joints of the robotic system, based on the planned motion;
characterizing a future magnetic field offset between the second ambient-field motor and the magnetic field of the MRI machine, during execution of the planned motion by the robotic system, based on the future pose;
calculating a future torque output limit of the second ambient-field motor, during execution of the planned motion by the robotic system, based on the future magnetic field offset and a current limit of the second ambient-field motor;
accessing a kinematic model that relates joint positions of the robotic system to three-dimensional positions of the set of joints;
estimating a future torque demand of the second ambient-field motor, during execution of the planned motion by the robotic system, based on the kinematic model of the robotic system and the planned motion; and
in response to the future torque demand of the second ambient-field motor approaching the future torque output limit of the second ambient-field motor, modifying the planned motion to:
define traversal of the first joint from the first joint position to a third joint position to drive the end effector along a second path, different from the first path, from the first end effector position to the second end effector position; and
increase a future margin between the future torque output limit and the future torque demand of the second ambient-field motor during execution of the planned motion by the robotic system.

15. The method of claim [0015]:
wherein driving the first ambient-field motor according to the first composite commutation angle comprises driving the first ambient-field motor according to the first composite commutation angle to hold the first joint in a first joint position; and
further comprising, at a second time succeeding the first time:
accessing a motor current amplitude of current supplied to the first ambient-field motor following the first time to hold the first joint in the first joint position; and
in response to the motor current amplitude approaching a current limit of the first ambient-field motor:
triggering a mechanical brake within the first joint to lock the first joint in the first joint position; and
reducing the motor current amplitude of current supplied to the first ambient-field motor.

16. The method of claim [0015]:
further comprising accessing a first imaging-state indicator indicating a non-imaging state of the MRI machine during the first time;
wherein characterizing the first magnetic field offset between the first ambient-field motor and the magnetic field of the MRI machine comprises:
based on the first imaging-state indicator, accessing a first magnetic field model representing magnetic field vectors within the bore of the MRI machine when the MRI machine occupies the non-imaging state; and
calculating the first magnetic field offset between the first ambient-field motor and a first magnetic field vector of the magnetic field of the MRI machine proximal the first ambient-field motor at the first time based on the first pose of the first ambient-field motor and the first magnetic field model; and
further comprising:
accessing a second set of joint positions of the set of joints at a second time;
accessing a second mechanical rotor position of the first rotor at the second time;
deriving a second pose of the first ambient-field motor within the MRI machine at the second time based on the second set of joint positions;
accessing a second imaging-state indicator indicating an imaging state of the MRI machine during the second time;
based on the second imaging-state indicator, accessing a second magnetic field model representing magnetic field vectors within the bore of the MRI machine when the MRI machine occupies the imaging state, the second magnetic field model different from the first magnetic field model;
calculating a second magnetic field offset between the first ambient-field motor and a second magnetic field vector of the magnetic field of the MRI machine proximal the first ambient-field motor at the second time based on the second pose of the first ambient-field motor and the second magnetic field model;
calculating a second composite commutation angle for the first ambient-field motor based on the second mechanical rotor position and the second magnetic field offset; and
driving the first ambient-field motor according to the second composite commutation angle.

17. The method of claim [0015]:
further comprising:
characterizing a torque constant of the first ambient-field motor at the first time based on the first magnetic field offset;
selecting a first set of control coefficients assigned to the first ambient-field motor based on the torque constant, the first set of control coefficients comprising a first proportional gain, a first integral gain, and a first derivative gain; and
generating a motor current command for the first ambient-field motor:
based on the first set of control coefficients; and
based on a position error of the first joint at the first time; and
wherein driving the first ambient-field motor according to the first composite commutation angle comprises driving the first ambient-field motor according to the first composite commutation angle and the motor current command.

18. The method of claim [0015], further comprising:
accessing a second set of joint positions of the set of joints at a second time succeeding the first time;
accessing a second mechanical rotor position of the first rotor at the second time;
deriving a second pose of the first ambient-field motor within the MRI machine at the second time based on the second set of joint positions;
calculating a second magnetic field offset between the first ambient-field motor and a second magnetic field vector of the magnetic field of the MRI machine proximal the first ambient-field motor at the second time based on the second pose of the first ambient-field motor;
calculating a second composite commutation angle for the first ambient-field motor based on the second mechanical rotor position and the second magnetic field offset;
calculating a revised second composite commutation angle, that limits a rate of change of composite commutation angle for the first ambient-field motor across the first time and the second time to reduce position oscillation of the first joint, based on the first composite commutation angle and the second composite commutation angle; and
driving the first ambient-field motor according to the revised second composite commutation angle.

19. A method comprising:
accessing a first set of joint positions of a set of joints, within a robotic system occupying a bore of an MRI machine, at a first time;
accessing a first mechanical rotor position of a first rotor, in a first ambient-field motor arranged within a first joint in the set of joints of the robotic system, at the first time;
deriving a first pose of the first ambient-field motor within the MRI machine at the first time based on the first set of joint positions;
calculating a first magnetic field vector representing a magnetic field within the bore of the MRI machine proximal the first ambient-field motor at the first time based on the first pose of the first ambient-field motor;
calculating a first electrical commutation offset for the first ambient-field motor based on the first magnetic field vector;
calculating a first composite commutation angle for the first ambient-field motor based on the first mechanical rotor position and the first electrical commutation offset; and
driving the first ambient-field motor according to the first composite commutation angle.

20. A method comprising:
accessing a first mechanical rotor position of a first rotor, in a first ambient-field motor arranged within a first joint in a set of joints of a robotic system occupying a bore of an MRI machine, at a first time;
accessing a first magnetic field vector of a magnetic field of the MRI machine proximal the first ambient-field motor at the first time;
calculating a first mechanical commutation angle for the first ambient-field motor based on the first mechanical rotor position;
calculating a first electrical commutation offset for the first ambient-field motor based on the first magnetic field vector;
calculating a first composite commutation angle for the first ambient-field motor based on the first mechanical commutation angle and the first electrical commutation offset; and
driving the first ambient-field motor according to the first composite commutation angle to traverse the first joint from a first position to a second position.
